(19) United States
(12) Patent Application Publication
(10) Pub. No.: US 2024/0156922 A1
(43) Pub. Date: May 16, 2024

(54) FUNGAL PROTEASE MIXTURES AND USES THEREOF

(71) Applicant: Bio-Cat, Inc., Troy, VA (US)

(72) Inventors: Sean Michael GARVEY, Zion Crossroads, VA (US); Kelly Marie TINKER, Columbia, VA (US); Justin Lamont GUICE, Haymarket, VA (US); Robert Daniel LITTLE, Charlottesville, VA (US); Christopher Edward SCHULER, Charlottesville, VA (US)

(21) Appl. No.: 18/552,254

(22) PCT Filed: Mar. 25, 2022
(86) PCT No.: PCT/US2022/022053

§ 371 Date: Sep. 25, 2023

Related U.S. Application Data
(60) Provisional application No. 63/166,188, filed on Mar. 25, 2021.

(51) Int. Cl. A61K 38/48; A61P 3/02

(52) U.S. Cl. CPC A61K 38/488; A61K 38/482; A61K 38/4886; A61P 3/02; C12Y 304/21063; C12Y 304/23018

(57) ABSTRACT

Fungal protease compositions, and more particularly, proteolytic enzyme mixtures comprising a plurality of Aspergillus proteases are provided. The disclosure also relates to protein hydrolysates, food and beverage products and dietary supplements produced using these proteolytic enzyme mixtures, and methods of making and using the same.

### CROSS-REFERENCE TO RELATED APPLICATIONS

This application claims benefit of U.S. Provisional Patent Application No. 63/166,188, filed Mar. 25, 2021, which is incorporated by reference herein in its entirety.

### TECHNICAL FIELD

Novel fungal protease compositions, and more particularly, proteolytic enzyme mixtures comprising a plurality of Aspergillus proteases are provided. The disclosure further relates to dietary supplements, foods, and beverage products containing these proteolytic enzyme mixtures or hydrolysates produced using these proteolytic enzyme mixtures, and methods of making and using the same.

### BACKGROUND

Protein is an essential dietary macronutrient that provides humans and animals with the amino acid building blocks for cells to synthesize hundreds of thousands of proteins. Intracellular proteins such as the actins and myosins in skeletal muscle support healthy cellular function and organismal physiology. Proteins are long chains of amino acids (or “AAs”) that are connected to each other by peptide bonds. At least 20 different amino acids can be encoded by a gene to direct the synthesis of a protein. Of these amino acids, 11 are non-essential amino acids that can be synthesized by human cells. The remaining 9 essential amino acids (or “EAAs”) are not made by human cells and must be supplied from the diet. EAAs include leucine, isoleucine, valine, histidine, lysine, methionine, phenylalanine, threonine, tryptophan. Of these 9 EAAs, leucine, isoleucine, and valine are also branched chain amino acids (or “BCAAs”), which are especially associated with skeletal muscle maintenance and growth. Dietary protein is hydrolyzed in the human gastrointestinal tract to produce peptides and free amino acids that are absorbed in to circulation and distributed to all cells of the body. Protein hydrolysis in the stomach is mediated by both hydrochloric acid and an endogenous protease called pepsin. Proteases are generally characterized as exopeptidases or endopeptidases depending on whether they cleave peptide bonds of the terminal amino acid or between internal amino acids of a peptide. Proteases may also be specific to a particular amino acid (or sequence of amino acids) on the substrate protein (or peptide) or nonspecific. Proteases are proteolytic enzymes that hydrolyze the peptide bonds between amino acids to release shorter peptides and free amino acids. In addition to gastric pepsin, the pancreas and small intestine also generate proteolytic enzymes that contribute to protein digestion and amino acid liberation.

Exogenous, oral enzyme supplementation is a candidate approach to optimize protein digestion and absorption of amino acids, in particular EAAs and BCAAs. Proteolytic enzymes accelerate the conversion of protein and peptides to amino acids. Dietary supplements containing proteolytic enzyme mixtures have been marketed to promote amino acid and BCAA liberation from dietary protein. Proteolytic enzyme mixtures known in the art include Aminogen®, a proteolytic enzyme preparation derived from A. oryzae and A. niger which comprises at least 2 proteolytic components as described in Oben, J., et al., “An Open Label Study to Determine the Effects of an Oral Proteolytic Enzyme System on Whey Protein Concentrate Metabolism in Healthy Males.” Journal of the International Society of Sports Nutrition. 2008. 5, 10, the entire contents of which is incorporated herein by reference. Proteolytic enzyme mixtures known in the art also include ProHydrolase®, a proteolytic enzyme preparation derived from Bacillus subtilis and Ananas comosus stem (i.e., pineapple) which comprises at least 2 proteolytic components as described in International Patent Application No. PCT/US2013/026657 (WO2014130007), and in Townsend, J. R., et al., “The Effect of ProHydrolase® on the Amino Acid and Intramuscular Anabolic Signaling Response to Resistance Exercise in Trained Males.” Sports (Basel). 2020. 8(2), 13, the entire contents of each of which are incorporated herein by reference.

The proper selection of an enzyme (or enzymes in a mixture) for oral dietary supplement use is important because proteolytic activity is affected by pH. Generally, the human stomach shows a pH value between 1.5 and 5.0, and the small intestine shows a pH value between 6.0 and 8.0. A proteolytic enzyme mixture may preferably contain enzymes that are optimally active across the entire pH gradient from stomach to small intestine. A proteolytic enzyme mixture may also preferably contain a balance of exopeptidase and endopeptidase activities.

Raw protein sources may be hydrolyzed to produce peptides and free amino acids using naturally occurring or recombinant proteolytic enzymes or by chemical decomposition. This hydrolysate may then be used for various purposes, e.g., as a seasoning for improved taste, food additive or dietary supplement for nutrition, or as a precursor or component of another protein-related product. Enzymatic digestion may proceed using a single proteolytic enzyme (e.g., a single, nonspecific exopeptidase that may gradually digest a given polypeptide or oligopeptide). Alternatively, digestion may involve the use of a mixture of proteases that display different proteolytic activity profiles (i.e., exopeptidase, endopeptidase, and combinations thereof). Proteolytic enzyme mixtures known in the art include Flavourzyme®, a proteolytic enzyme preparation derived from A. oryzae which comprises at least 5 proteolytic components as described in International Patent Application No. PCT/DK1994/000165 (WO1994/025580), and in Merz, M., et al., “Flavourzyme, an Enzyme Preparation with Industrial Relevance: Automated Nine-step Purification and Partial Characterization of Eight Enzymes.” Journal of Agricultural and Food Chemistry. 2015. 63(23), 5682-5693, the contents of each of which is incorporated herein by reference. The proper selection of an enzyme (or enzymes in a mixture) for production of a hydrolysate is important because the characteristics and properties of the hydrolysate will vary depending on the type and degree of proteolysis. For example, incomplete digestion may generate a hydrolysate enriched in oligopeptides or free amino acids which create a bitter taste or chalky mouthfeel, resulting in a product unsuitable for certain purposes (e.g., an additive for food products). Other properties of proteolytic enzymes, such as stability, efficiency, cost, and compatibility with other common solvents and reagents are also relevant to the selection of a proteolytic enzyme or mixture of enzymes for hydrolysate production. These limitations constrain the commercial or industrial use of particular enzymes and combinations thereof.

### Summary of Various Embodiments

In a general aspect, the present disclosure relates to combinations of proteases obtained from members of the genus Aspergillus, such as A. oryzae or A. melleus, which are capable of digesting protein from various sources. In some exemplary aspects, the proteolytic enzyme mixtures described herein may be used as ingredients in dietary supplements, protein powders, or foods to promote protein digestion, to promote post-prandial plasma amino acid levels, or both, following consumption of a dietary supplement, food, beverage, or meal. In some exemplary aspects, the proteolytic enzyme mixture described herein may be used to produce a hydrolysate containing free EAAs and free BCAAs. In some exemplary aspects, the proteolytic enzyme mixtures described herein may be used to produce a hydrolysate that is more easily digested, more easily absorbed, or both, by the gastrointestinal system of a human or animal. In some exemplary aspects, the proteolytic enzyme mixtures described herein may be also used to produce a hydrolysate that has improved flavor and/or mouthfeel compared to a hydrolysate prepared using currently available enzymes that often produce bitter and/or chalky hydrolysates. Moreover, the mixtures of enzymes described herein are stable and maintain activity over a broad range of temperatures and pH levels, providing additional options for commercial and industrial applications.

In some aspects, the proteolytic enzyme mixtures comprise 1) a Fungal Protease A (a 42 kDa protease with exo- and endo-protease activity obtained from A. oryzae; CAS No. 9025-49-4; IUBMB Enzyme Commission (EC) No. 3.4.23.18), 2) Fungal Protease AM (a 34 kDA protease with peptidase activity obtained from A. melleus; CAS No. 9074-07-1; EC No. 3.4.11.-), and 3) a Fungal Protease A2 (a 35 kDa fungal neutral protease obtained from A. oryzae; CAS No. 9025-49-4; EC No. 3.4.24.-), when analyzed by polyacrylamide gel electrophoresis. In other aspects, the proteolytic enzyme mixture comprises SEQ ID NOs: 1, 2, and 3.

In other general aspects, the proteolytic enzyme mixture may be used as an ingredient in protein supplements and oral nutritional supplements, wherein the proteolytic enzyme mixture is prepared as a powder and may be dry-blended with protein in a manufacturing process that yields a protein or oral nutritional supplement.

In other general aspects, the disclosure provides methods of administering a dietary supplement comprising the disclosed proteolytic enzyme mixtures to increase amino acids, EAA and/or BCAA absorption following consumption of a dietary supplement, beverage, food, or meal.

In other general aspects, the disclosure provides methods of preparing a protein hydrolysate from various protein sources using the disclosed proteolytic enzyme mixtures, and in particular, protein hydrolysates containing amino acids, EAAs, and/or BCAAs.

In still other general aspects, food products, additives, dietary supplements and beverages comprising the protein hydrolysates described herein are provided.

Methods of using the disclosed dietary supplements containing the proteolytic enzyme mixture or protein hydrolysates are also provided, including methods of increasing exercise performance, decreasing muscle breakdown during exercise, improving recovery from exercise, or combinations thereof, by administering a dietary supplement containing the proteolytic enzyme mixture or protein hydrolysate prepared described herein, alone or as part of a food product, dietary supplement, or beverage.

Additional aspects will be readily apparent to one of skill in light of the totality of the disclosure.

### BRIEF DESCRIPTION OF THE DRAWINGS

FIG. 1A is a graph illustrating the relative activity of Fungal Protease A across various pH levels.

FIG. 1B is a graph illustrating the relative activity of Fungal Protease A across various temperature levels.

FIG. 2A is a graph illustrating the relative activity of Fungal Protease AM across various pH levels.

FIG. 2B is a graph illustrating the relative activity of Fungal Protease AM across various temperature levels.

FIG. 3A is a graph illustrating the relative activity of Fungal Protease A2 across various pH levels.

FIG. 3B is a graph illustrating the relative activity of Fungal Protease A2 across various temperature levels.

FIG. 3C is a graph illustrating the residual activity of Fungal Protease A2 across various pH levels.

FIG. 3D is a graph illustrating the residual activity of Fungal Protease A2 across various temperature levels.

FIG. 4 is a graph illustrating the relative activity of OPTIZIOME™ P³ HYDROLYZER™ (also referred to as P³ HYDROLYZER™), Aminogen®, and ProHydrolase®, across a range of pH levels.

FIG. 5A is a graph illustrating the relative activity of OPTIZIOME™ P³ HYDROLYZER™, across a range of pH levels.

FIG. 5B is a graph illustrating the relative activity of OPTIZIOME™ P³ HYDROLYZER™ across various temperature levels.

FIG. 6 is a bar chart that illustrates a comparative analysis of the proteolytic activities of Aminogen®, ProHydrolase®, and P³ HYDROLYZER™ based on the release of 20 different amino acids following simulated gastric digestion of whey protein for 60 minutes.

FIG. 7 is a bar chart that illustrates a comparative analysis of the proteolytic activities of ProHydrolase® (60 minutes), P³ HYDROLYZER™ (15 minutes), and Aminogen® (60 minutes) based on the release of 20 different amino acids following simulated gastric digestion of whey protein.

FIG. 8 is a bar chart that illustrates a comparative analysis of the proteolytic activities of Aminogen®, ProHydrolase®, and P³ HYDROLYZER™, based on the release of 3 different BCAAs following simulated gastric digestion of whey, soy, pea, and rice proteins for 60 minutes.

FIGS. 9A-9D are charts illustrating a comparative analysis of the proteolytic activities of Aminogen®, ProHydrolase®, and P³ HYDROLYZER™, based on the release of total amino acids (FIG. 9A), EAAs (FIG. 9B), BCAAs (FIG. 9C), and leucine (FIG. 9D) following simulated gastric digestion of whey protein for 60 minutes.

FIGS. 10A-10D are charts illustrating a comparative analysis of the proteolytic activities of Aminogen®, ProHydrolase®, and P³ HYDROLYZER™, based on the release of total amino acids (FIG. 10A), EAAs (FIG. 10B), BCAAs (FIG. 10C), and leucine (FIG. 10D) following simulated gastric digestion of soy protein for 60 minutes.

FIGS. 11A-11D are charts illustrating a comparative analysis of the proteolytic activities of Aminogen®, ProHydrolase®, and P³ HYDROLYZER™, based on the release of total amino acids (FIG. 11A), EAAs (FIG. 11B), BCAAs (FIG. 11C), and leucine (FIG. 11D) following simulated gastric digestion of pea protein for 60 minutes.

FIGS. 12A-12D are charts illustrating a comparative analysis of the proteolytic activities of Aminogen®, ProHydrolase®, and P³ HYDROLYZER™, based on the release of total amino acids (FIG. 12A), EAAs (FIG. 12B), BCAAs (FIG. 12C), and leucine (FIG. 12D) following simulated gastric digestion of rice protein for 60 minutes.

FIGS. 13A-D are charts illustrating a comparative analysis of the proteolytic activities of Aminogen®, ProHydrolase®, and P³ HYDROLYZER™, showing differences in the concentrations of leucine (FIG. 13A), BCAAs (FIG. 13B), EAAs (FIG. 13C), and total amino acids (FIG. 13D) produced from whey protein following simulated salivary-gastric digestion 122 minutes per the INFOGEST protocol. *, p<0.05; **, p<0.01; ***, p<0.001; ****, p<0.0001.

FIGS. 14A-D are charts illustrating a comparative analysis of the proteolytic activities of Aminogen®, ProHydrolase®, and P³ HYDROLYZER™, showing differences in the concentrations of leucine (FIG. 14A), BCAAs (FIG. 14B), EAAs (FIG. 14C), and total amino acids (FIG. 14D) produced from pea protein following simulated salivary-gastric digestion for 122 minutes per the INFOGEST protocol. ****, p<0.0001.

FIGS. 15A-D are charts illustrating a comparative analysis of the proteolytic activities of Aminogen®, ProHydrolase®, and P³ HYDROLYZER™, showing differences in the concentrations of leucine (FIG. 15A), BCAAs (FIG. 15B), EAAs (FIG. 15C), and total amino acids (FIG. 15D) produced from soy protein following simulated salivary-gastric digestion for 122 minutes per the INFOGEST protocol. ***, p<0.001; ****, p<0.0001.

FIG. 16 is a graph illustrating the results of a flavor preference test comparing assessors' preference for whey protein shakes treated with either P³ HYDROLYZER™ or ProHydrolase®.

FIG. 17A-D are graphs illustrating the plasma concentrations of leucine (FIG. 17A), BCAAs (FIG. 17B), EAAs (FIG. 17C), and total amino acids (FIG. 17D) across the first 2 hours of a clinical aminoacidemia trial before and following consumption of a pea protein shake with placebo or P³ HYDROLYZER™. Each circle represents a blood draw for amino acid analysis. Black circle, placebo; white circle, P³ HYDROLYZER™ (n=24 subjects, cross-over design).

FIG. 18A-B are charts illustrating the total area under the curve (AUC) plasma concentrations of EAAs (FIG. 18A) and total amino acids (FIG. 18B) across the first 2 hours of a clinical aminoacidemia trial before and following consumption of a pea protein shake with placebo or P³ HYDROLYZER™ (n=24 subjects, cross-over design).

### DETAILED DESCRIPTION OF VARIOUS EMBODIMENTS

The present disclosure relates to proteolytic enzyme mixtures comprising a plurality of fungal proteases obtained from members of the genus Aspergillus (e.g., from A. oryzae and A. melleus). These proteolytic enzyme mixtures may be administered to a subject as a dietary supplement (e.g., to improve protein digestion or the absorption of amino acids, EAAs, and/or BCAAs). They may also be used to produce a protein hydrolysate containing amino acids, EAAs and/or BCAAs, which may also possess additional beneficial properties (e.g., less bitterness, improved flavor and/or mouthfeel) compared to protein hydrolysate produced using currently available proteases and proteolytic enzyme mixtures. Additionally, methods of using these proteolytic enzyme mixtures, and various products (e.g., foods, beverages, dietary supplements) and other vehicles for administering the proteolytic enzyme mixtures or resulting protein hydrolysate are also provided. Methods of producing protein hydrolysates enriched with amino acids, EAAs, and/or BCAAs from a single protein source (e.g., without the need for supplementation with additional amino acids, EAAs and/or BCAAs from another source) are also provided.

Proteins are high molecular weight polymers composed of multiple amino acids linked by peptide bonds. These bonds must be cleaved in order for protein to be absorbed and utilized by a human or other organism, with such cleavage typically being performed by endogenous proteolytic enzymes of the gastrointestinal tract that separate the polypeptides into its constituent free amino acids. Amino acids may be classified as essential or non-essential for any given organism, depending on whether an organism is capable of synthesizing the given amino acid. For a dietary regimen to be considered adequate for the support of normal physiological functions, it should contain all essential amino acids in the appropriate levels and in proper proportions. For humans, the nine essential amino acids are leucine, isoleucine, valine, methionine, tryptophan, phenylalanine, threonine, lysine and histidine.

Three of the essential amino acids (valine, leucine and isoleucine) have aliphatic side-chains with a branch, i.e., a central carbon atom bound to three or more carbon atoms. These BCAAs are particularly notable because these amino acids are an important nutritional factor for proper muscle physiology and metabolism. Reports further indicate that athletic and exercise performance and recovery may be improved by BCAA supplements (See e.g., Glynn, E. L., et al., “Excess Leucine Intake Enhances Muscle Anabolic Signaling but Not Net Protein Anabolism in Young Men and Women.” The Journal of Nutrition. 2010. 140(11), 1970-1976; Sharp, C. P. M., et al., “Amino Acid Supplements and Recovery from High-Intensity Resistance Training.” Journal of Strength and Conditioning Research. 2010. 24(4), 1125-1130; Foure, A. & Bendahan, D. Is Branched-chain Amino Acids Supplementation an Efficient Nutritional Strategy to Alleviate Skeletal Muscle Damage? A Systematic Review. Nutrients. 2017. 9(10), 1047). In older adults, who are at risk of severe muscle wasting, BCAA supplementation in combination with exercise has also been shown to enhance performance and muscle strength (See e.g., Ikeda, T., et al. Effects and Feasibility of Exercise Therapy Combined with Branched-chain Amino Acid Supplementation on Muscle Strengthening in Frail and Pre-Frail Elderly People Requiring Long-term Care: A Crossover Trial. Applied Physiology, Nutrition, and Metabolism. 2016. 41(4), 438-445). The remaining non-essential amino acids provide a source of metabolizable nitrogen required for the biosynthesis of proteins, purines, nucleic acids, and other metabolites.

In view of the above, there has been commercial interest in dietary supplements and food additives that contain EAAs and BCAAs (e.g., protein powders and energy drinks directed to athletes). These supplements are typically prepared by fermentation of a raw protein source (e.g., whey protein), or by digestion of a raw protein source using a proteolytic enzyme or combination of enzymes and then supplementing the end product with BCAAs or other EAAs obtained from a second process or source. The manufacturing of such products is therefore complicated by the fact that amino acids must typically be obtained from multiple sources and mixed together to obtain a product which has the desired profile and ratios (e.g., enriched in BCAAs and/or EAAs).

The present disclosure provides proteolytic enzyme mixtures that simplify this process by generating protein hydrolysates already enriched in EAAs, and more particularly BCAAs. Use of these proteolytic enzyme mixtures reduces the complexity and manufacturing costs associated with having to obtain amino acids from different sources. Moreover, protein hydrolysates produced using these proteolytic enzyme mixtures have been found to have an improved taste, texture (e.g., mouthfeel) and solubility profiles compared to hydrolysates produced using known proteolytic enzymes and combinations thereof.

Protein hydrolysates produced using the present methods are therefore well-suited for use in commercial food products, dietary supplements, additives, and beverages. These food products and other vehicles may in turn be used by consumers, and athletes in particular, to provide nutrition, as well as athletic and/or exercise benefits.

### Proteolytic Enzyme Mixtures Comprising a Plurality of Aspergillus Proteases

In one general aspect, the present disclosure provides a proteolytic enzyme mixture comprising a plurality of fungal proteases obtained from members of the genus Aspergillus (e.g., a combination of at least three proteases obtained from A. oryzae or A. melleus). One or more of these enzymes may possess exopeptidase, endopeptidase and/or peptidase activity, alone or operating in combination with other enzymes in the mixture. In some exemplary aspects, the mixture has proteolytic activity across a pH range spanning from 3.0 to 9.0, or any range of integer values therein. In some embodiments, the relative activity of the proteolytic enzyme mixture will be >40% across a pH range of 4.0 to 9.0, >60% across a pH range of 5.0 to 9.0, >80% across a pH range of 5.7 to 6.3, and/or >90% across a pH range of 5.8 to 6.2, measured at 60° C. The relative activity may also be >40% across a temperature range of 20 to 80° C., >60% across a temperature range of 40 to 80° C., >80% across a temperature range of 55 to 70° C., and/or >90% across a temperature range of 56 to 63° C., measured at pH 6.0. The proteolytic enzyme mixture may be capable of digesting a raw protein source (e.g., plant protein, whey protein) and producing a protein hydrolysate enriched in EAAs and/or BCAAs when applied to a food product or dietary supplement comprising a protein (e.g., a protein shake containing whey isolate as the protein) at a minimum of 1,000 to 150,000 hemoglobin unit tyrosine base (HUT) units per gram of the protein. As used herein, references to HUT units of the enzymes and enzyme mixtures described herein are calculated using the standard protocol labeled “PROTEOLYTIC ACTIVITY, FUNGAL (HUT)” in the Food Chemicals Codex (FCC), 12. Ed. (published Mar. 1, 2020), the contents of which is hereby incorporated by reference in its entirety. Briefly, one HUT unit (HUT/g) is defined as the amount of enzyme that produces a hydrolysate from bovine hemoglobin at pH 4.7 whose absorbance at 275 nanometers is the same as that of a solution containing 1.10 μg/mL of tyrosine in 0.006 N hydrochloric acid. Increased HUT correlates with increased proteolytic activity of a protease or proteolytic mixture.

In some aspects, the proteolytic enzyme mixture may alternatively be applied to a food product or dietary supplement at a minimum of 1,000 to 150,000 HUT units per gram of total protein in the composition (e.g., at a minimum of 10,000; 20,000; 30,000; 40,000; 50,000; 60,000; 70,000; 80,000; 90,000; 100,000; 110,000; 120,000; 130,000; 140,000; or 150,000 HUT/g, or alternatively within a range bounded by any of these values). In some exemplary aspects, the proteolytic enzyme mixture comprises a three-enzyme blend and may produce a hydrolysate with amino acid, EAA, and/or BCAA enrichment at a concentration several-fold larger than the amino acid, EAA, and/or BCAA concentrations of a hydrolysate prepared by digestion with only one or two of the three enzymes in the proteolytic enzyme mixture. As such, the proteolytic enzyme mixture may be used to improve the absorption of amino acids (e.g., EAAs, BCAAs) by a human or animal that consumes a treated food product or dietary supplement comprising the proteolytic enzyme mixture, by increasing the amount of free amino acids, EAAs and/or BCAAs released during digestion.

For example, in some aspects, the proteolytic enzyme mixture may comprise “OPTIZIOME™ P³ HYDROLYZER™” (also referred to as “p³” or “P³ HYDROLYZER™” herein), a mixture of three fungal proteases distributed by BIO-CAT, Inc.: 1) Fungal Protease A (a 42 kDa protease with exo- and endo-protease activity obtained from A. oryzae; CAS No. 9025-49-4; IUBMB Enzyme Commission (EC) No. 3.4.23.18), present at 25,000 HUT units/g, 2) Fungal Protease AM (a 34 kDA protease with peptidase activity obtained from A. melleus; CAS No. 9074-07-1; EC No. 3.4.11.-), present at 2,500 HUT units per gram, and 3) Fungal Protease A2 (a 35 kDa fungal neutral protease obtained from A. oryzae; CAS No. 9025-49-4; EC No. 3.4.24.-), present at 100,000 HUT units per gram. The molecular weight of the enzymes is analyzed by polyacrylamide gel electrophoresis. As described herein, this combination has been shown to release amino acids, EAAs, and BCAAs across a wide range of pH and temperature levels (e.g., pH 3 to 9, and a temperature of 20-80° C.) while maintaining high activity.

In other aspects, the proteolytic enzyme mixture comprises a plurality of fungal proteases from the genus Aspergillus, wherein the mixture comprises SEQ ID NO: 1, a protease with peptidase activity obtained from A. melleus (CAS No. 9074-07-1; EC No. 3.4.11.-), and a fungal neutral protease obtained from A. oryzae (CAS No. 9025-49-4; EC No. 3.4.24.-). In another aspect, the mixture comprises SEQ ID NO: 1, SEQ ID NO: 2, and a fungal neutral protease obtained from A. oryzae (CAS No. 9025-49-4; EC No. 3.4.24.-). In another aspect, the mixture comprises SEQ ID NO: 1, a protease with peptidase activity obtained from A. melleus (EC No. 3.4.11.-), and SEQ ID NO: 3.

In another aspect, the mixture comprises a protease with exo- and endo-protease activity obtained from A. oryzae (CAS No. 9025-49-4; EC No. 3.4.23.18), SEQ ID NO: 2, and a fungal neutral protease obtained from A. oryzae (CAS No. 9025-49-4; EC No. 3.4.24.-). In another aspect, the mixture comprises a protease with exo- and endo-protease activity obtained from A. oryzae (CAS No. 9025-49-4; EC No. 3.4.23.18), SEQ ID NO: 2, and SEQ ID NO: 3.

In another aspect, the mixture comprises a protease with exo- and endo-protease activity obtained from A. oryzae (CAS No. 9025-49-4; EC No. 3.4.23.18), a protease with peptidase activity obtained from A. melleus (CAS No. 9074-07-1; EC No. 3.4.11.-), and SEQ ID NO: 3.

In other aspects, the proteolytic enzyme mixture comprises a plurality of fungal proteases from the genus Aspergillus, wherein the mixture comprises SEQ ID NO: 1, a 34 kDA protease with peptidase activity obtained from A. melleus (CAS No. 9074-07-1; EC No. 3.4.11.-), and a 35 kDA fungal neutral protease obtained from A. oryzae (CAS No. 9025-49-4; EC No. 3.4.24.-), wherein the molecular weight of the proteases are analyzed by polyacrylamide gel electrophoresis. In another aspect, the mixture comprises SEQ ID NO: 1, SEQ ID NO: 2, and a 35 kDa fungal neutral protease obtained from A. oryzae (CAS No. 9025-49-4; EC No. 3.4.24.-) when analyzed by polyacrylamide gel electrophoresis. In another aspect, the mixture comprises SEQ ID NO: 1, a 34 kDA protease with peptidase activity obtained from A. melleus (CAS No. 9074-07-1; EC No. 3.4.11.-) when analyzed by polyacrylamide gel electrophoresis, and SEQ ID NO: 3.

In another aspect, the mixture comprises a 42 kDa protease with exo- and endo-protease activity obtained from A. oryzae (CAS No. 9025-49-4; EC No. 3.4.23.18), SEQ ID NO: 2, and a 35 kDa fungal neutral protease obtained from A. oryzae (CAS No. 9025-49-4; EC No. 3.4.24.-), wherein the molecular weight of the proteases are analyzed by polyacrylamide gel electrophoresis. In another aspect, the mixture comprises a 42 kDa protease with exo- and endo-protease activity obtained from A. oryzae (CAS No. 9025-49-4; EC No. 3.4.23.18) when analyzed by polyacrylamide gel electrophoresis, SEQ ID NO: 2, and SEQ ID NO: 3.

In another aspect, the mixture comprises a 42 kDa protease with exo- and endo-protease activity obtained from A. oryzae (CAS No. 9025-49-4; EC No. 3.4.23.18), a 34 kDA protease with peptidase activity obtained from A. melleus (CAS No. 9074-07-1; EC No. 3.4.11.-), and SEQ ID NO: 3, wherein the molecular weight of the proteases are analyzed by polyacrylamide gel electrophoresis.

In other aspects, the proteolytic enzyme mixture comprises a plurality of fungal proteases from the genus Aspergillus, wherein the mixture comprises SEQ ID NO: 1, SEQ ID NO: 2, and SEQ ID NO: 3.

In some aspects, the proteolytic enzyme mixture comprises a plurality of fungal proteases from the genus Aspergillus, wherein the mixture comprises SEQ ID NO: 1, SEQ ID NO: 2, and/or SEQ ID NO: 3, or a fragment or variant of any of these enzymes. For example, in some aspects, the proteolytic enzyme mixture comprises at least one variant enzyme which shares at least 90, 91, 92, 93, 94, 95, 96, 97, 98 or 99% full-length sequence identity with any one of SEQ ID NOs: 1-3, and which retains one or more of the enzymatic activities of SEQ ID NOs: 1-3. For example, a polypeptide sequence may differ from any one of SEQ ID NOs:1-3 by the presence of one or more conservative or non-conservative substitutions which do not impact the catalytic or other activity of the enzyme. As used herein, the term “sequence identity” refers to the degree to which two polypeptide sequences are identical (i.e., on a residue-by-residue basis) over the window of comparison. The percentage of sequence identity is calculated by comparing two optimally aligned sequences over the window of comparison, determining the number of positions at which identical residues occurs in both sequences to yield the number of matched positions, dividing the number of matched positions by the total number of positions in the window of comparison (i.e., the window size), and multiplying the result by 100 to yield the percentage of sequence identity.

In some aspects, the proteolytic enzyme mixture comprises a plurality of fungal proteases from the genus Aspergillus, wherein the mixture comprises at least one enzyme which shares at least 90, 91, 92, 93, 94, 95, 96, 97, 98 or 99% full-length sequence identity with a portion of the sequence of SEQ ID NOs: 1-3 (e.g., with the region spanning positions 78-404 of SEQ ID NO: 1 or with the region spanning positions 246-634 of SEQ ID NO: 3, which are predicted to represent the mature forms of the enzymes represented by these SEQ ID NOs). In some aspects, the proteolytic enzyme mixture comprises a plurality of fungal proteases from the genus Aspergillus, wherein the mixture comprises at least one enzyme which shares at least 90, 91, 92, 93, 94, 95, 96, 97, 98 or 99% full-length sequence identity with the region spanning positions 21-404 of SEQ ID NO: 1 or with the region spanning positions 19-634 of SEQ ID NO: 3.

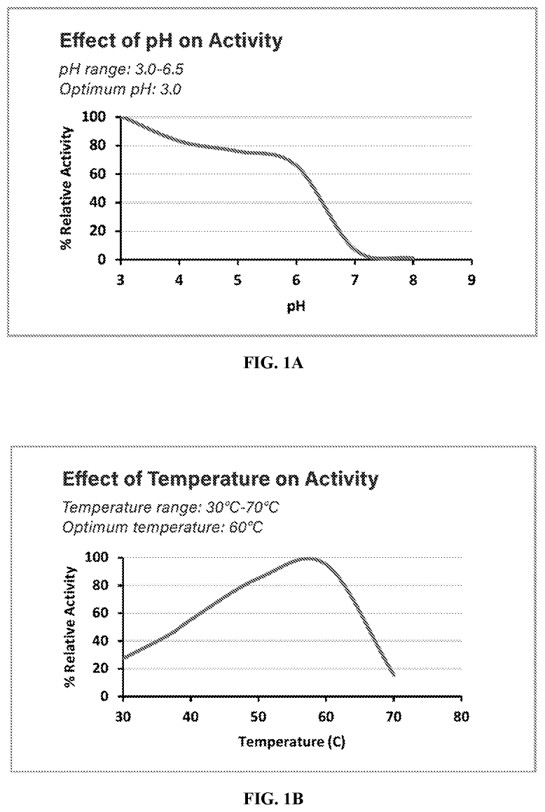

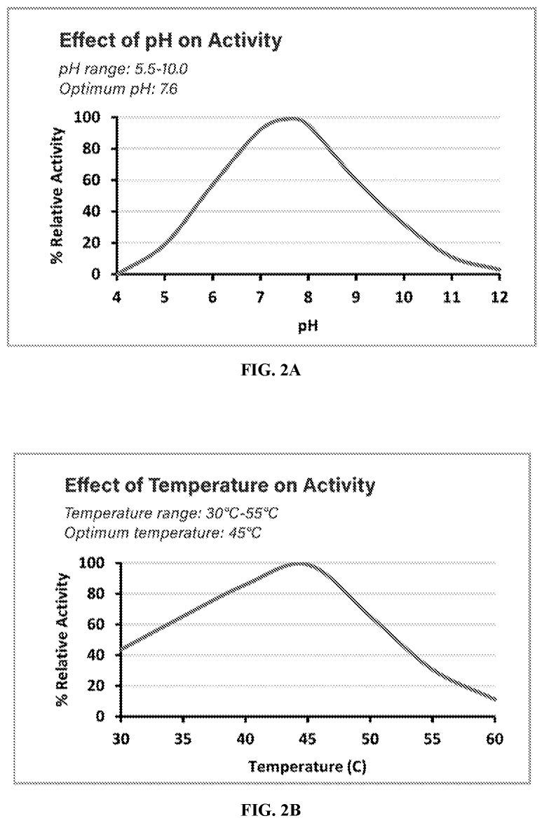

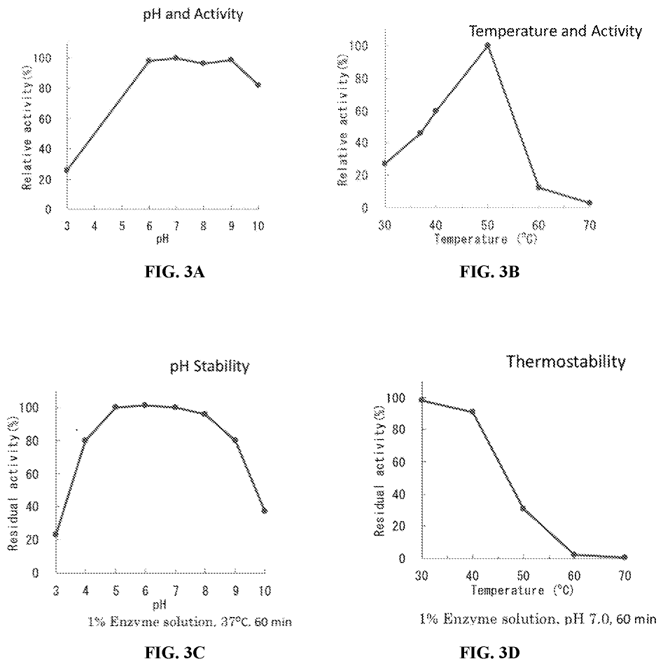

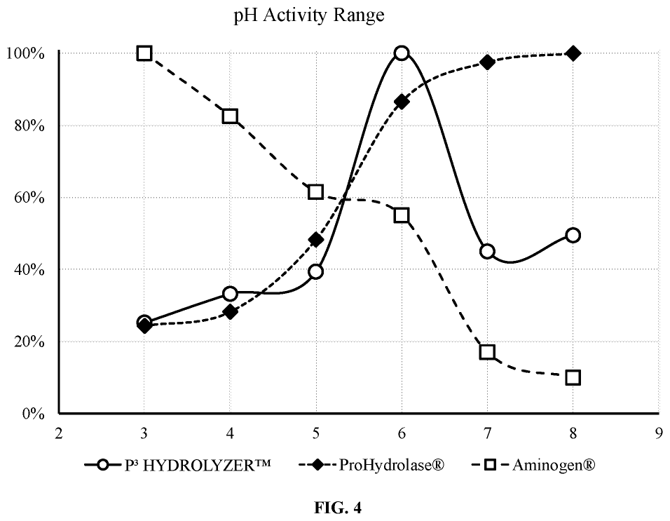

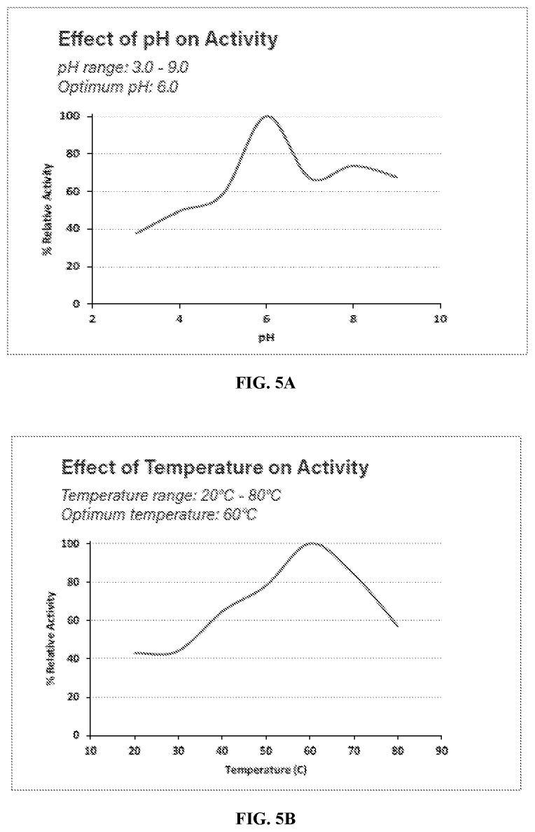

FIGS. 1A-1B provide graphs that illustrate the relative activity levels of Fungal Protease A across various temperature and pH levels. FIGS. 2A-2B provide graphs that illustrate the relative activity levels of Fungal Protease AM across various temperature and pH levels. FIGS. 3A-3D provide graphs that illustrate the relative and residual activity levels of Fungal Protease A2 across various temperature and pH levels. FIG. 4 is a graph that illustrates the relative activity levels of P³ HYDROLYZER™, ProHydrolase® and Aminogen® across various pH levels. As shown by this figure, P³ HYDROLYZER™ displays peak activity at approximately pH 6. However, it retains activity across a broad range of pH values spanning pH 3 to pH 8, a range of pH common across the gastric and intestinal compartments of the mammalian digestive tract. The activity profile of P³ HYDROLYZER™ under various pH and temperature levels is further illustrates by FIGS. 5A-5B, which graph the activity level of P³ HYDROLYZER™ from pH 3 to 9 and across a span of 20-80° C.

While the assay results described herein pertain to P³ HYDROLYZER™, it is understood that in some alternative aspects, a proteolytic enzyme mixture according to the disclosure may include at least two of the enzymes in P³ HYDROLYZER™ or comprise at least two of SEQ ID NOs: 1, 2, or 3 (e.g., SEQ ID NOs: 1 and 2, 1 and 3, or 2 and 3). It is further understood that the amounts or ratios of the enzymes in the proteolytic enzyme mixture may be varied to produce a mixture having enhanced or reduced activity levels. For example, Fungal Protease A, Fungal Protease AM, and Fungal Protease A2 may be combined at a ratio of approximately 10:1:40, 10:1:20, 10:1:75, 3:1:10, as measured in HUT activity units, or any other ratio which provides a desired activity level as measured in HUT units. In some aspects, the ratio of any of the individual components may vary by ±5, 10, 15, 20, 25, 30, 35, 40, 45, or 50% from any of the foregoing examples or ratios described herein (e.g., the ratio may be 5-15:0.5-1.5:10-30, as measured in HUT activity units).

In practice, P³ HYDROLYZER™ will typically be used at 1,000-2,000 HUT unit per gram of protein being digested (e.g., approximately 1,300 HUT units). However, it is understood that this amount will be varied subject to routine optimization for a given application (e.g., additional HUTs/g may be necessary at a lower incubation temperature for a given protein). Additional enzymes (e.g., proteases), coenzymes, cofactors, solvents, salts, etc., may be added to any of the protease enzyme mixtures disclosed herein as desired to improve or modify the digestion process as necessary or desired for a particular implementation.

The proteolytic enzyme mixtures described herein may be in a dehydrated, powdered, granular or freeze-dried form. In other aspects, the enzyme mixture is cell-free.

### Dietary Supplements Comprising Proteolytic Enzyme Mixtures

In some aspects, proteolytic enzyme mixtures described herein may be formulated as dietary supplements, protein supplements, and/or nutritional supplement compositions that may be administered to a subject to provide one or more benefits (e.g., to increase protein digestion and/or the absorption of amino acids, EAAs, or BCAAs, or to improve the subject's muscle health). As used herein, the term “dietary supplement” refers to a manufactured product taken by mouth that comprises one or more “dietary ingredients” intended to supplement the diet (i.e., food) of a subject. Exemplary dietary ingredients include proteins, amino acids, carbohydrates, fat, vitamins, minerals, metabolites, probiotics, enzymes, herbs and botanicals. Unlike medicaments, dietary supplements are not intended to treat, diagnose, prevent, or cure diseases. A dietary supplement may comprise, e.g., a proteolytic enzyme mixture as described herein and at least 10, 20, 30, 40, 50, 60, 70, 80, or 90 wt. % of a dietary ingredient or a mixture of dietary ingredients. In some aspects, the dietary ingredient portion comprises a wt. % within a range bounded by any of these values (e.g., 10-20 wt. %). A “protein supplement” is a type of dietary supplement comprising at least 10 dry wt. % protein, wherein the amount of protein in the composition is greater than that of either carbohydrate or fat. A “nutritional supplement” is a type dietary supplement comprising protein, carbohydrates, and fat.

In accordance with certain embodiments disclosed herein, the proteolytic enzyme mixtures contains at least 0.01%, 0.02%, 0.03%, 0.04%, 0.05%, 0.06%, 0.07%, 0.08%, 0.09%, 0.10%, 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, or 30% by weight solids of a protease preparation from Aspergillus oryzae, including at least 40%, at least 50%, at least 60%, at least 70%, at least 80%, and at least 90% by weight solids of a protease preparation from Aspergillus oryzae. In accordance with certain of the preceding embodiments, the proteolytic enzyme mixture contains 30% to 100% total HUT activity from the Aspergillus oryzae protease preparation, including from 45% to 100%, 50% to 100%, 70% to 100%, 80% to 100%, and 90% to 100% total HUT activity of the proteolytic enzyme mixture.

In some aspects, dietary supplements, protein supplements, and/or nutritional supplement compositions comprising a proteolytic enzyme mixture according to the disclosure may be suitable for oral administration. Oral administration, as defined herein, includes any form of administration in which the composition including the proteolytic enzyme mixture passes through the esophagus of the subject. For example, oral administration typically refers to oral consumption, but may also include administration through nasogastric intubation, in which a tube is run from the nose to the stomach of the subject to administer the composition. Oral administration is a form of enteral administration (i.e., administration through the digestive tract). Other forms of enteral administration suitable for use with the methods disclosed herein include administration through a gastric or jejunal tube. In accordance with the embodiments described herein, suitable forms of the composition for enteral administration to the subject include caplets, tablets, pills, capsules, chewable tablets, quick dissolve tablets, effervescent tablets, solutions, suspensions, emulsions, multi-layer tablets, bi-layer tablets, soft gelatin capsules, hard gelatin capsules, lozenges, chewable lozenges, beads, granules, particles, microparticles, dispersible granules, sachets, and combinations thereof.

In some aspects, dietary supplement, protein supplement, and/or nutritional supplement compositions may be formulated consisting of or consisting essentially of a proteolytic enzyme mixture according to the disclosure. In other aspects, the proteolytic enzyme mixture is formulated into a protein supplement. Such protein supplements disclosed herein are useful to provide supplemental sources of protein, including providing the subjects one or more benefits as described herein. In other embodiments, the proteolytic enzyme mixture is formulated in to a nutritional supplement. Such nutritional supplements disclosed herein are useful to provide supplemental sources of nutrition, including providing the subjects one or more benefits as described herein.

In accordance with certain methods of the embodiments disclosed herein, the dietary supplements, protein supplements, and nutritional supplement compositions may be provided as needed to deliver the desired level of proteolytic enzyme mixture, e.g., by providing at least one serving per day to achieve the desired effect. In some aspects, the dietary supplements, protein supplements, and nutritional supplement compositions maybe administered at one serving per day, two servings per day, three servings per day, four servings per day, etc., as needed to achieve a desired effect. Typically, the compositions disclosed herein are administered in at least one serving per day or at least two servings per day.

In some aspects, the proteolytic enzyme mixture is administered as a dietary supplement in capsule form before, during, or following consumption of a protein supplement. The final dose per serving of the proteolytic enzyme mixture may be between 5,000 and 300,000 HUT, 10,000 and 100,000 HUT, and 25,000 and 75,000 HUT. Table 1 shows an example formulation, from which a 250 mg capsule would be expected to deliver ˜31,000 HUT. Table 2 shows an alternative exemplary formulation for a 300 mg capsule.

TABLE 1
An exemplary formulation of a dietary supplement
in capsule form comprising a proteolytic enzyme
mixture according to the disclosure.

| | AMOUNT | ACTIVITY |
|---|---|---|
| INGREDIENTS | (mg/1,000 mg) | (HUT/g) |
| Maltodextrin | 687.3 | n/a |
| Fungal Protease A2 | 256.4 | 100,000 |
| (Aspergillus oryzae) | | |
| Fungal Protease A | 34.4 | 25,000 |
| (Aspergillus oryzae) | | |
| Fungal Protease AM | 21.9 | 2,500 |
| (Aspergillus melleus) | | |

TABLE 2
A second exemplary formulation of a dietary supplement
in capsule form comprising a proteolytic enzyme
mixture according to the disclosure.

| | AMOUNT | ACTIVITY |
|---|---|---|
| INGREDIENTS | (mg) | (HUT/g) |
| Maltodextrin | 171.8 | n/a |
| Fungal Protease A2 | 64.1 | 25,000 |
| (Aspergillus oryzae) | | |
| Microcrystalline Cellulose | 25 | n/a |
| Fungal Protease A | 8.6 | 6,250 |
| (Aspergillus oryzae) | | |
| Fungal Protease AM | 5.5 | 625 |
| (Aspergillus melleus) | | |
| Silicon Dioxide | 2.3 | n/a |
| Magnesium Stearate | 1.2 | n/a |

In some aspects of the disclosure, the proteolytic enzyme mixture is administered as a dietary supplement in the form of a powder sachet or stick pack reconstituted in 4 to 6 ounces of water before, during, or following consumption of a meal. The final dose per serving of the proteolytic enzyme mixture may be between 5,000 and 300,000 HUT, 10,000 and 100,000 HUT, and 25,000 and 75,000 HUT. Table 3 shows an example formulation, from which a 4.5 g stick pack would be expected to deliver ˜31,000 HUT:

TABLE 3
An exemplary formulation of a dietary supplement
in powder sachet or stick pack form comprising
a protein mixture according to the disclosure.

| | AMOUNT PER |
|---|---|
| INGREDIENTS | SERVING (g) |
| Hydrolyzed Collagen | 4.0 |
| Maltodextrin | 0.376 |
| Fungal Protease A2 | 0.064 |
| (Aspergillus oryzae) | |
| Stevia Leaf Extract | 0.030 |
| Strawberry Flavor | 0.015 |
| Fungal Protease A | 0.009 |
| (Aspergillus oryzae) | |
| Fungal Protease AM | 0.006 |
| (Aspergillus melleus) | |

In some aspects of the disclosure, the proteolytic enzyme mixture is formulated as a protein supplement. The final dose per serving of the proteolytic enzyme mixture is between 5,000 and 300,000 HUT, 10,000 and 100,000 HUT, and 25,000 and 75,000 HUT. Tables 4 and 5 show example protein powder formulations, from which a serving would be expected to deliver ˜31,000 HUT:

TABLE 4
An exemplary formulation of a protein supplement comprising
a protein mixture according to the disclosure.

| | AMOUNT PER |
|---|---|
| INGREDIENTS | SERVING (g) |
| Whey protein concentrate | 25 |
| Cocoa powder | 2.7 |
| Maltodextrin | 1 |
| Natural Flavors | 0.6 |
| Xantham Gum | 0.1 |
| Carageenan | 0.1 |
| Salt | 0.1 |
| Fungal Protease A2 | 0.064 |
| (Aspergillus oryzae) | |
| Sucralose | 0.030 |
| Acesulfame potassium | 0.025 |
| Fungal Protease A | 0.009 |
| (Aspergillus oryzae) | |
| Fungal Protease AM | 0.006 |
| (Aspergillus melleus) | |

TABLE 5
A second exemplary formulation of a protein supplement comprising
a protein mixture according to the disclosure.

| | AMOUNT PER |
|---|---|
| INGREDIENTS | SERVING (g) |
| Pea Protein Isolate | 11 |
| Rice Protein | 9 |
| Coconut Oil | 2.0 |
| Tapioca Maltodextrin | 1 |
| Natural Vanilla Flavors | 0.6 |
| Guar Gum | 0.1 |
| Carageenan | 0.1 |
| Sea Salt | 0.1 |
| Stevia Leaf Extract | 0.08 |
| Fungal Protease A2 | 0.064 |
| (Aspergillus oryzae) | |
| Fungal Protease A | 0.009 |
| (Aspergillus oryzae) | |
| Fungal Protease AM | 0.006 |
| (Aspergillus melleus) | |

Any source of protein may be used so long as it is suitable for protein supplement or nutritional supplement compositions and is otherwise compatible with any other selected ingredients or features in the protein supplement or nutritional supplement compositions. For example, the source of protein may include, but is not limited to, intact, hydrolyzed, and partially hydrolyzed protein, which may be derived from any known or otherwise suitable source such as milk (e.g., casein, whey), animal (e.g., meat, fish, egg), cereal (e.g., rice, corn, oat, wheat), vegetable (e.g., pea, soy, hemp, potato), pulses (chick pea, mung bean, fava bean), fungi, bacteria, insect and combinations thereof. The source of protein may also include a mixture of amino acids known for use in protein supplements or a combination of such amino acids with the intact, hydrolyzed, and partially hydrolyzed proteins described herein. The amino acids may be naturally occurring or synthetic amino acids. The amino acids may include branched chain amino acids, essential amino acids, non-essential amino acids, or combination thereof.

Examples of suitable sources of protein for use in the protein supplements and nutritional supplements disclosed herein include, but are not limited to, whey protein concentrates, whey protein isolates, whey protein hydrolysates, acid caseins, sodium caseinates, calcium caseinates, potassium caseinates, casein hydrolysates, milk protein concentrates, milk protein isolates, milk protein hydrolysates, nonfat dry milk, condensed skim milk, pea protein isolates, pea protein hydrolysates, soy protein concentrates, soy protein isolates, soy protein hydrolysates, pea protein concentrates, collagen proteins, potato proteins, rice proteins, insect proteins, earthworm proteins, fungal (e.g., mushroom) proteins, proteins expressed by microorganisms (e.g., bacteria and algae), and the like, as well as combinations thereof. The nutritional supplement compositions can include any individual source of protein or a combination of two or more the various sources of protein listed above or otherwise encompassed by the general inventive concepts.

A variety of dairy protein and plant protein sources may be utilized for the protein system of the protein supplement or nutritional supplement described herein. An exemplary dairy protein suitable for use in the nutritional supplement powder described herein is Avonlac® 282, a whey protein concentrate, available from Glanbia Nutritionals (Kilkenny, Ireland). An exemplary plant protein suitable for use in the nutritional supplement powder described herein is NUTRALYS® S85F, a pea protein isolate, available from Roquette Freres (Lestrem, France).

In some aspects of the disclosure, the proteolytic enzyme mixture is formulated in to a nutritional supplement. The final dose per serving of the proteolytic enzyme mixture may be between 5,000 and 300,000 HUT, 10,000 and 100,000 HUT, and 25,000 and 75,000 HUT. Table 6 shows an example nutritional formulation, from which a serving would be expected to deliver ˜31,000 HUT:

TABLE 6
An exemplary formulation of a nutritional supplement comprising
a protein mixture according to the disclosure.

| | AMOUNT PER |
|---|---|
| INGREDIENTS | SERVING (g) |
| Corn Maltodextrin | 20.0 |
| Sugar | 10.0 |
| Milk protein concentrate | 10.0 |
| Soybean Oil | 5.0 |
| Soy Protein Isolate | 5.0 |
| Canola Oil | 3.0 |
| Vitamin and Mineral Blend | 1.5 |
| Whey Protein Concentrate | 1.0 |
| Guar Gum | 1.0 |
| Carageenan | 1.0 |
| Salt | 0.8 |
| Natural Flavors | 0.6 |
| Fungal Protease A2 | 0.064 |
| (Aspergillus oryzae) | |
| Sucralose | 0.030 |
| Acesulfame potassium | 0.025 |
| Fungal Protease A | 0.009 |
| (Aspergillus oryzae) | |
| Fungal Protease AM | 0.006 |
| (Aspergillus melleus) | |
| Alpha-galactosidase | 0.003 |

The dietary supplements, protein supplements and nutritional supplements described herein may be administered to a subject in order to increase protein digestion and/or to improve the absorption of amino acids, EAAs, and/or BCAAs. In other aspects, these compositions may be administered to a subject to improve muscle health. In each case, such methods comprise administering at least one serving per day of a composition comprising a proteolytic enzyme mixture according to the disclosure. In some aspects, such methods comprise administering 10 mg to 1,000 mg of proteases per serving, or approximately 5,000 HUT to 300,000 HUT per serving, to the subject.

### Protein Hydrolysate Compositions and Methods of Preparation

Proteolytic enzyme mixtures described herein may be used to produce protein hydrolysates enriched in essential amino acids and/or BCAAs compared to protein hydrolysates produced by other protease enzymes and mixtures known in the art. Such hydrolysates may be produced from any raw protein source capable of digestion by a selected proteolytic enzyme mixture, including plant proteins (e.g., soy, hemp, rice, whey or pea protein), animal proteins (e.g., beef, chicken, or pork) and microbial proteins. Non-traditional protein sources such as insect protein (e.g., cricket protein) may also be used, as may proteins expressed from a recombinant organism (e.g., protein synthesized by a genetically-modified yeast culture).

As described above, proteolytic enzyme mixtures according to the disclosure may be used, in some embodiments, to produce hydrolysates having desirable properties such as enriched levels of essential amino acids or BCAAs. In some exemplary aspects, hydrolysates produced as described herein may have free leucine, isoleucine and/or valine levels which are several-fold higher than the levels of these free amino acids in protein hydrolyzed by any of the individual enzymes in the proteolytic enzyme mixture or by currently available proteases and mixtures. In some embodiments, such hydrolysates may be produced as a one-step process without supplementation from a secondary amino acid source (e.g., the initial hydrolysate may have a several-fold increase in one or more of these amino acids, avoiding the need for supplementation with additional BCAAs). In some exemplary aspects, hydrolysates produced as described herein may contain at least 10, 20, 30 or 40 mg/L of valine, at least 10, 20, 30 or 40 mg/L of isoleucine, and/or at least 10, 20, 30 or 40 mg/L of leucine. In some instances, the concentration of leucine in such hydrolysates may be further enriched to a level of at least 50, 100 or 150 mg/L.

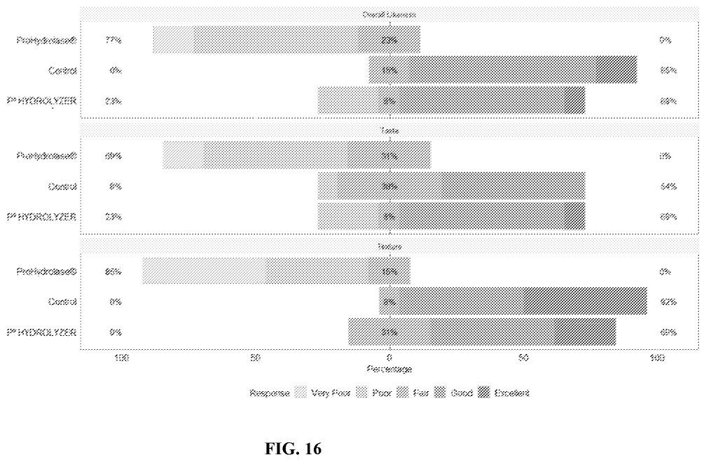

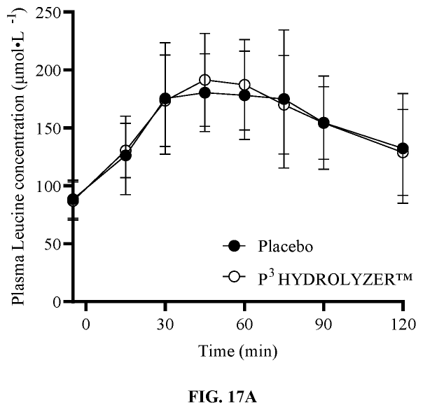

In addition to providing higher concentrations of free BCAAs, hydrolysates produced according to the disclosure have also been found to display improved organoleptic properties. As noted above, hydrolysates produced according to methods known in the art often have a chalky mouthfeel and/or bitter taste. However, as described by Example 4 and illustrated by FIG. 16, protein hydrolysates prepared using P³ HYDROLYZER™ were preferred by a panel of assessors compared to untreated whey protein shake or a hydrolysate digested with ProHydrolyase®.

### Food Products, Ingredients or Additives, Dietary Supplements and Beverages Comprising Protein Hydrolysates

Protein hydrolysates produced according to the present disclosure may be used as food products, dietary supplements, as an ingredient or additive for a food product, in beverages, or in any other vehicle suitable for administration to or ingestion by a person or animal. In particular, hydrolysates according to the disclosure enriched in essential amino acids and/or BCAAs may be particularly desirable as food products, beverages or dietary supplements intended for athletes and subjects interested in improving exercise performance.

In some exemplary aspects, a protein hydrolysate may be prepared from a protein source (e.g., plant, animal or microbial-sourced raw protein) using any of the protease enzyme mixtures or methods of production described herein. The resulting hydrolysate may be optionally processed, such as by heat-inactivating the protease enzymes used to perform the digestion, chemically treating the mixture, and/or by filtering the mixture. The hydrolysate may also be optionally converted into a form more convenient for transport or storage (e.g., by drying, dehydrating or freeze-drying the hydrolysate). The hydrolysate may, subject to any such optional processing, be added to a food product, dietary supplement, beverage or any other vehicle suitable for administration to a human or animal, as indicated above.

In some exemplary aspects, the hydrolysate is dried or dehydrated to form a protein powder enriched in essential amino acids and/or BCAAs. In other exemplary aspects, the hydrolysate is added to a food product such as a meal replacement or energy bar or beverage. The hydrolysate may be added to a vehicle as a powder or in liquid form, as is preferred for a given application.

### Methods of Using Protein Hydrolysates

Protein hydrolysates may be provided or administered to a human or animal in need of additional nutrition and/or to promote or provide a beneficial physiological effect. Protein hydrolysates enriched in essential amino acids and/or BCAAs are particularly useful as these classes of amino acid are associated with proper nutrition, muscle physiology and metabolism. As a result, protein hydrolysates produced according to the methods described herein may be used as a dietary supplement or as part of a treatment for humans or animals in order to improve nutrition or to improve athletic or exercise performance.

In some exemplary aspects, a protein hydrolysate as described herein may be administered to a subject in need thereof once, on a periodic basis or as part of any other regimen suitable to provide the subject with sufficient levels of one or more essential amino acids and/or BCAAs (e.g., to provide a desirable trait or reach a selected threshold associated with a desirable physiological state). The hydrolysate may be provided or administered as a food product, additive or ingredient to a food product, dietary supplement, beverage, or any other vehicle suitable which allows a subject to ingest or otherwise absorb amino acids in the hydrolysate.

Protein hydrolysates prepared using P³ HYDROLYZER™ in accordance with any of the exemplary aspects above may be provided to a human or animal to promote nutrition or improved athletic or exercise performance, particularly hydrolysates enriched in BCAAs. It is understood that any such hydrolysates may be provided to a human or animal in need thereof as part of a food product, dietary supplement or beverage and may be provided in any amount necessary to provide a desirable function or outcome, with such amounts being the product of routine optimization depending on the nature of the individual or animal receiving the hydrolysate and/or the composition of the hydrolysate.

All statements herein reciting principles, aspects, and embodiments of the invention as well as specific examples thereof, are intended to encompass both structural and functional equivalents thereof. Additionally, it is intended that such equivalents include both currently known equivalents and equivalents developed in the future, i.e., any elements developed that perform the same function, regardless of structure. The scope of the present invention, therefore, is not intended to be limited to the exemplary embodiments shown and described herein. Rather, the scope and spirit of present invention is embodied by the appended claims.

All publications and patents cited in this specification are herein incorporated by reference as if each individual publication or patent were specifically and individually indicated to be incorporated by reference and are incorporated herein by reference to disclose and describe the methods and/or materials in connection with which the publications are cited. The citation of any publication is for its disclosure prior to the filing date and should not be construed as an admission that the present invention is not entitled to antedate such publication by virtue of prior invention. Further, the dates of publication provided may be different from the actual publication dates which may need to be independently confirmed.

### EXAMPLES

The following examples demonstrate the performance of an exemplary protease mixture comprising Fungal Protease (A. oryzae), Fungal Protease AM (A. melleus), and Fungal Protease A2 (A. oryzae) at a ratio of 10:1:40. Each of these enzyme ingredients was obtained from BIO-CAT, Inc (Troy, Virginia, USA).

### Example 1: Comparative Amino Acid Releasing Performance of P³ HYDROLYZER™ in a 60 Minute Simulation of Gastric Digestion of Whey Protein

The primary objective of these in vitro experiments was to determine the effects of P³ HYDROLYZER™ in releasing amino acids from dietary proteins in a gastric digestion simulation, with a comparison to two additional protease ingredient products. To start, the effects of P³ HYDROLYZER™ on amino acid release from whey protein powder (NAKED Nutrition®; Coral Gables, FL, USA) was compared to Aminogen® and ProHydrolase®, two commercially available protease mixtures. Three experimental treatment groups, in addition to one control with no supplemental enzymes, were tested: 1) a blend of proteases and maltodextrin that meets at least the minimum protease concentration in the Aminogen® product; 2) the ProHydrolase® blend of proteases; and 3) a blend of P³ HYDROLYZER™ and maltodextrin. Three grams of whey protein (approximately 1/10 serving size) were dissolved in 20 mL deionized water for each experimental enzyme treatment and control. Enzymes were weighed on tared weigh paper and then transferred to 20 mL test tubes using deionized water. Enzymes were prepared as follows: 1) 0.250 grams of a protease blend and maltodextrin that meets at least the minimum protease concentration in Aminogen® per 10 mL, 2) 0.250 grams ProHydrolase® per 5 mL, and 3) 0.125 grams of a protease blend and maltodextrin reflective of the final protease concentration in P³ HYDROLYZER™ per 10 mL. All treatments reflect an equivalent recommended dose of the enzymes in Aminogen®, ProHydrolase®, and P³ HYDROLYZER™. One mL of each enzyme solution (approximately 1/10 recommended dose) was added to a separate beaker of dissolved protein. One mL deionized water (instead of enzyme) was also added to a separate beaker of dissolved protein (control). 2.5 mL simulated, acidic gastric solution (0.015 g mucin, 0.0125 grams pepsin 1:10,000, electrolytes, pH 1.9) was added to each beaker, including the control. This simulated gastric solution has been previously described (See Donhowe, E., et al., “Characterization and In Vitro Bioavailability of 0-Carotene: Effects of Microencapsulation Method and Food Matrix.” LWT—Food Science and Technology. 2014. 57, 42-48, which is hereby incorporated by reference in its entirety). Importantly, the simulated gastric solution contains porcine pepsin, which is an endogenous, naturally occurring mammalian protease that is released in to the stomach during food or beverage consumption. This porcine pepsin in this in vitro experiment simulates the activity of human gastric pepsin. The experimental samples now containing protein, experimental enzymes (with the exception of the control), and simulated gastric fluid with pepsin were then placed on a stir plate in a 37° C. water bath for 60 minutes with agitation by magnetic stir bar. After the full 60 minute incubation, beaker contents—now called “gastric digestas”—were transferred to 50 mL centrifuge tubes and enzymatic activity was halted by placing tubes in a 90° C. water bath for 10 minutes. Heat-killed gastric digestas were stored at 4° C. until high performance liquid chromatography (HPLC) analysis of all 20 essential and non-essential amino acids using o-phthalaldehyde (OPA) and 9-fluorenylmethyl chloroformate (FMOC). The results reported here are the average of amino acid values measured from three gastric simulations carried out on three separate days (n=3). A second experiment was also performed using the same compositions and parameters, except that the simulated gastric digestion incubation time was reduced to 15 minutes to better understand early activity of P³ HYDROLZYER™ during gastric digestion.

Amino acids were measured by HPLC (Agilent 1100 Series HPLC, Agilent Technologies, Inc.; Santa Clara, CA, USA) with fluorescence detection. Combining OPA and FMOC enables fast pre-column derivatization of amino acids for chromatographic analysis. The HPLC reaction mixture was buffered at a pH of 10.2, which allows direct derivatization of acid hydrolyzed protein/peptide samples. Primary amino acids contain both a basic amino group and an acidic carboxyl group. Proline is the only proteinogenic amino acid that is a secondary amino acid, i.e., the nitrogen atom is attached both to the α-carbon and to a chain of three carbons that together form a five-membered ring. Only primary amino acids react with OPA, so FMOC is needed for the detection of proline. The primary amino acids were reacted first with OPA using 3-mercaptopropionic acid (3-MPA). The secondary are then derivatized using FMOC. The incorporation of 3-MPA into the indoles decreases their hydrophobicity, and as a result, the OPA derivatives elute chromatographically before the FMOC derivatives. Excess FMOC and its degradation products elute after the last of the secondary amino acids and do not interfere with the analysis.

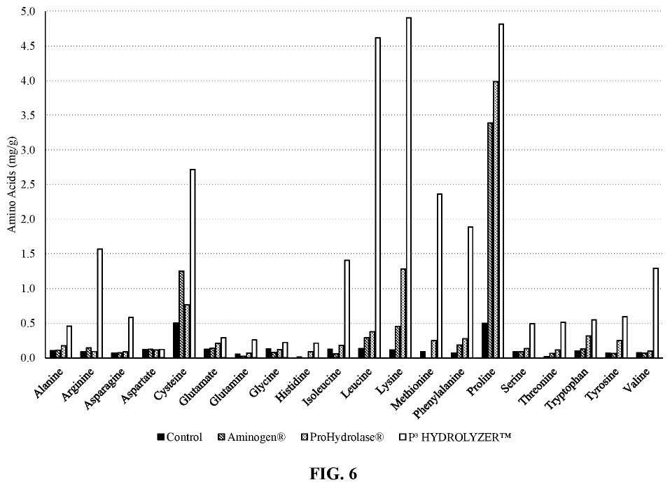

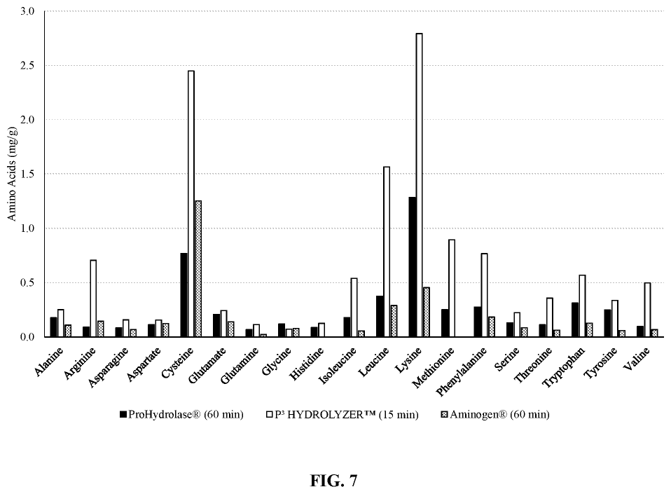

The results of these experiments are summarized by FIGS. 6-7. As demonstrated by these experiments, P³ HYDROLYZER™ displayed superior performance at both time points (i.e., after a 15 minute and 60 minute digestion), releasing >2-fold greater levels of several amino acids from whey protein compared to Aminogen® and ProHydrolase®. FIG. 7 illustrates that only 15 minutes of digestion with P³ HYDROLYZER™ released several amino acids at greater levels than the full 60 minutes of digestion with Aminogen® or ProHydrolase®. Physiologically, it takes approximately 0.5 to 4 hours, depending on the amount of food or beverage consumed, to reach maximum postprandial total plasma amino acid concentrations after protein consumption. As evidenced by these data, P³ HYDROLYZER™ may be applied to (or ingested concurrently with) a food product, beverage product, or dietary supplement containing protein to expedite the digestive process and to increase the amount of free amino acids that are available for absorption during and following digestion. Under in vivo conditions, it is expected that this increase in the amount of free amino acids will result in increased uptake by gut cells, higher blood amino acid concentrations, and increased absorption by tissues and organs such as skeletal muscle, which requires amino acids for the synthesis of new muscle proteins and muscle growth.

### Example 2: Comparative Amino Acid Releasing Performance of P³ HYDROLYZER™ in a 60 Minute Simulation of Gastric Digestion of Whey, Soy, Pea, and Rice Proteins

A similar experiment was performed to evaluate the use of P³ HYDROLYZER™ to release BCAAs from several additional protein sources (i.e., whey, soy, pea and rice protein), compared to a control sample with protein and only porcine pepsin, and protein digested with ProHydrolase® and Aminogen®. One dairy protein source and three plant protein sources were evaluated for their amino acid release profile under experimental enzymatic treatment: 1) whey protein powder (NAKED Nutrition®; Coral Gables, FL, USA), 2) soy protein isolate powder (Hard Eight Nutrition LLC, Hendersonville, NV, USA), 3) pea protein isolate powder (Hard Eight Nutrition LLC, Hendersonville, NV, USA), and 4) brown rice powder (Raw Power®, Coeur d'Alene, ID, USA).

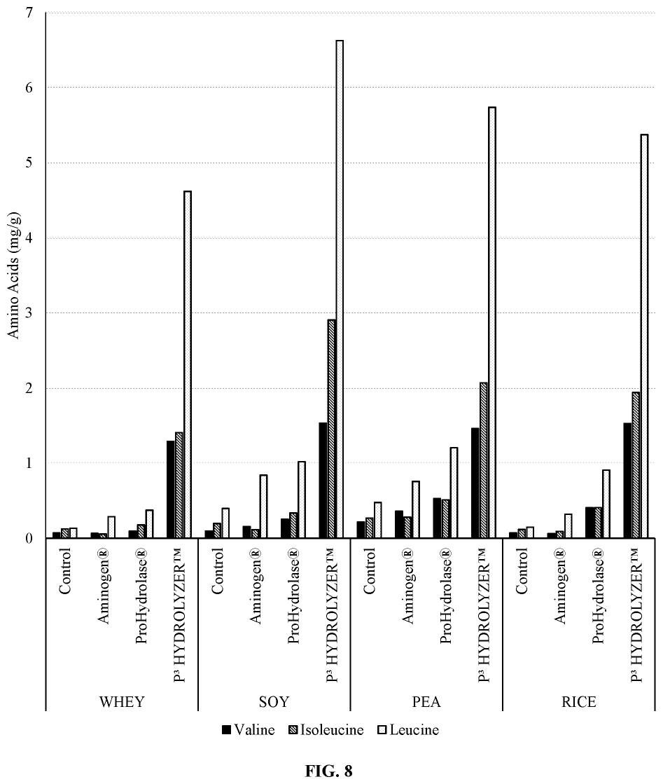

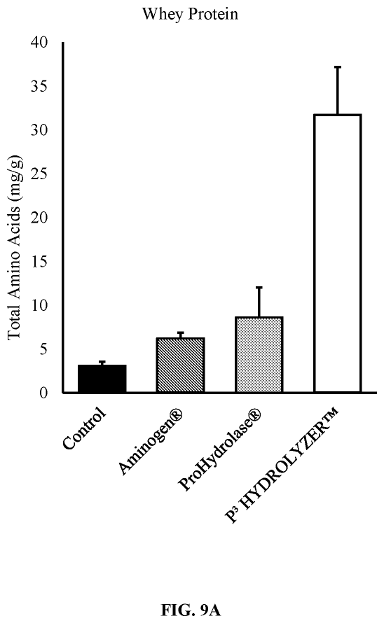

For each protein source, 3 treatment groups in addition to no enzyme control were tested: 1) a blend of proteases and maltodextrin that meets at least the minimum protease concentration in the Aminogen® product; 2) the ProHydrolase® blend of proteases; and 3) a blend of P³ HYDROLYZER™ and maltodextrin. The test compositions with experimental enzymes, protein, and simulated gastric fluid (containing porcine pepsin) were prepared, digested for 60 minutes, and analyzed as described in Example 1. The results of this experiment are summarized by FIG. 8, which provides a graph showing the concentration of BCAAs released by each enzyme-protein substrate pairing. As illustrated by FIG. 8, the gastric digestas treated with P³ HYDROLYZER™ are remarkably enriched in BCAAs compared to the digestas treated with ProHydrolase® and Aminogen®.

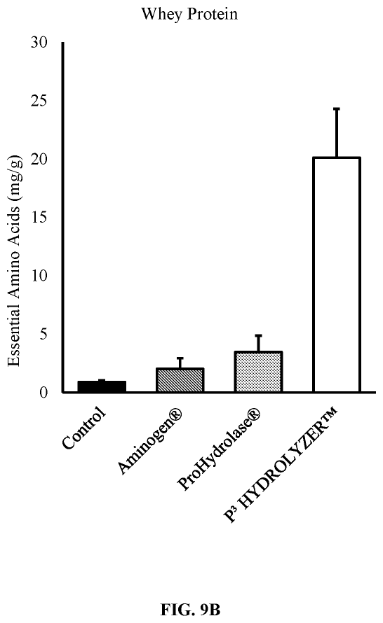

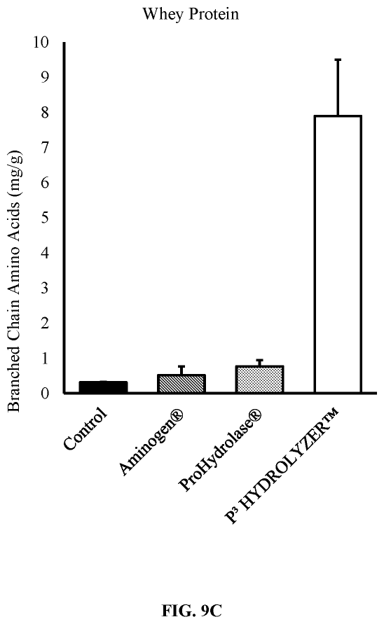

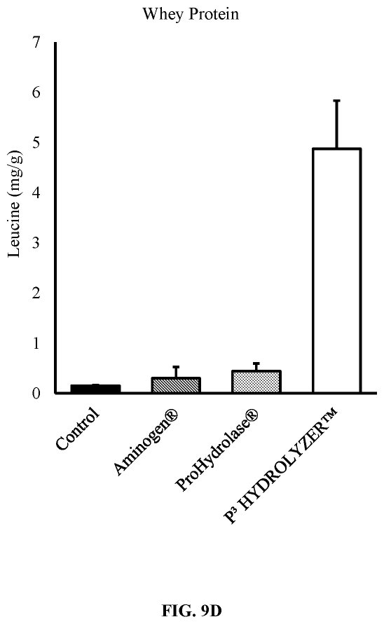

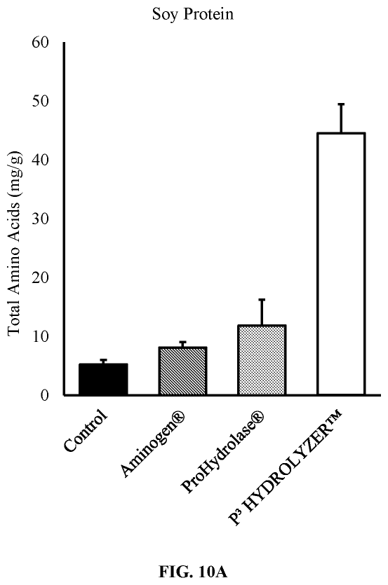

The effects of P³ HYDROLYZER™ on total amino acid, EAA, and leucine release profiles, in addition to the BCAA concentrations reported previously in Example 2, were also determined. The results of these additional measurements are summarized by FIGS. 9-12, which provide graphs showing the concentration of AAs, EAAs, BCAAs and leucine released from whey, soy, pea, and rice protein sources by P³ HYDROLYZER™, compared to two competitor protease mixtures and a control (protein and pepsin only). As illustrated by FIGS. 9-12, the gastric digestas treated with P³ HYDROLYZER™ across all 4 protein sources are remarkably enriched in total amino acids, EAAs, BCAAs and leucine, as compared to the gastric digestas treated with Aminogen® and ProHydrolase®.

To better characterize the superior performance of P³ HYDROLYZER™, a statistical analysis was performed using R® version 3.6.2 (R Core Team, 2020). Graphs were produced using the gglot2 package (Wickham, 2016). A one-way ANOVA analysis was performed to determine significance and an F-statistic of p<0.05 was considered statistically significant. Tukey's Honestly Significant Difference adjustment was used for multiple comparisons using a 95% family-wise confidence level. Each individual amino acid, total EAAs, total BCAAs, and total amino acids were analyzed.

P³ HYDROLYZER™ treatment of whey protein promoted greater amino acid liberation compared to competitor protease products, including greater liberation of total amino acids, EAAs BCAAs and leucine (FIG. 9, Table 7; all comparisons to P³ HYDROLYZER™ were statistically significant). These data in Table 7 represent values from averaging the amino acid liberation across 3 separate experiments performed on 3 separate days. Adjusted values are the result of subtracting the control values from the experimental enzyme treatment values to establish normalized baseline values adjusted to controls. Adjusted values are compared vs. P³ HYDROLYZER™ to determine fold-change and percent increases. The “Average” value represents the average of the two competitor enzyme ingredient products, Aminogen® and ProHydrolase®. The “Fold Change” value represents the average of the two competitor enzyme ingredient products, rounded down to the nearest whole number. “Unadjusted” values do not subtract the control from the experimental enzyme treatments.

TABLE 7
Amino acid release from whey protein in a 60 minute simulation of gastric
digestion (Comp, competitor; Comp A, Aminogen ®; Comp B, ProHydrolase ®).

| | | | | | | | |
|---|---|---|---|---|---|---|---|
| Measured | Adjusted | | P³ vs | | | | |
| Value | Values | P³ vs | Comp | Fold | P³ vs Comp | Unadjusted | Unadjusted |
| (mg/g) | (mg/g) | Comp | Average | Change | Unadjusted | average | fold change |

Essential amino acids:

| | | | | | | | | |
|---|---|---|---|---|---|---|---|---|
| Control | 0.89 | | | | | 2257% | | 22x |
| Comp A | 2.01 | 1.12 | 1714% | | | 1000% | | |
| Comp B | 3.44 | 2.55 | 753% | 1234% | 12x | 584% | 792% | 7x |
| P³ | 20.09 | 19.2 | | | | | | |

Branched chain amino acids:

| | | | | | | | | |
|---|---|---|---|---|---|---|---|---|
| Control | 0.31 | | | | | 2545% | | 25x |
| Comp A | 0.51 | 0.2 | 3790% | | | 1547% | | |
| Comp B | 0.76 | 0.45 | 1684% | 2737% | 27x | 1038% | 1293% | 12x |
| P³ | 7.89 | 7.58 | | | | | | |

Total amino acids:

| | | | | | | | | |
|---|---|---|---|---|---|---|---|---|
| Control | 3.1 | | | | | 1023% | | 10x |
| Comp A | 6.2 | 3.1 | 923% | | | 511% | | |
| Comp B | 8.6 | 5.5 | 520% | 721% | 7x | 369% | 440% | 4x |
| P³ | 31.7 | 28.6 | | | | | | |

Leucine:

| | | | | | | | | |
|---|---|---|---|---|---|---|---|---|
| Control | 0.15 | | | | | 3247% | | 32x |
| Comp A | 0.30 | 0.15 | 3147% | | | 1623% | | |
| Comp B | 0.44 | 0.29 | 1628% | 2387% | 23x | 1107% | 1365% | 13x |
| P³ | 4.87 | 4.72 | | | | | | |

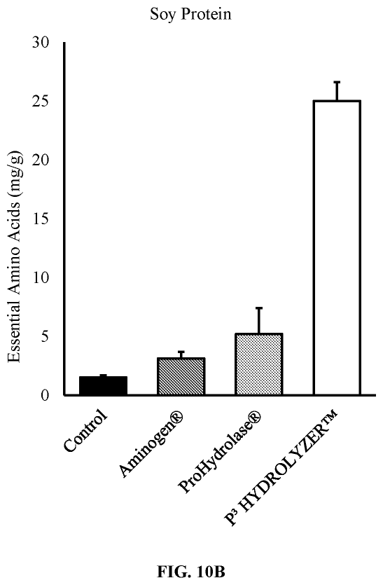

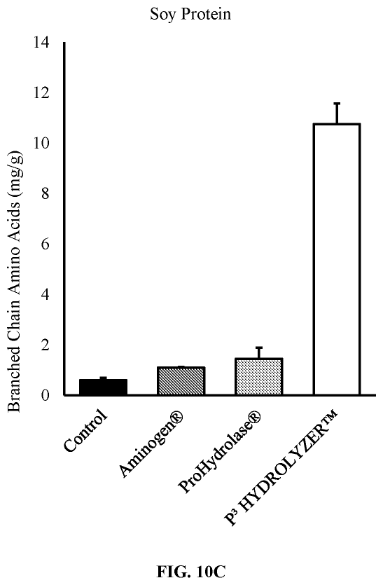

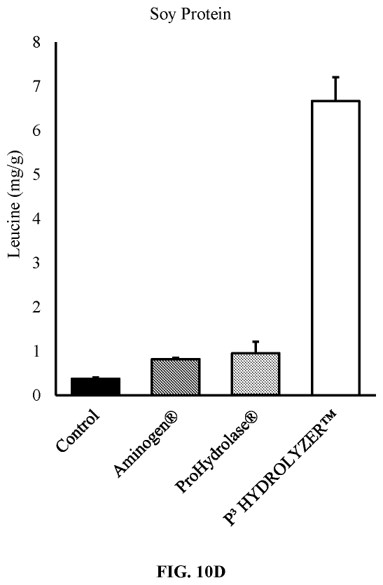

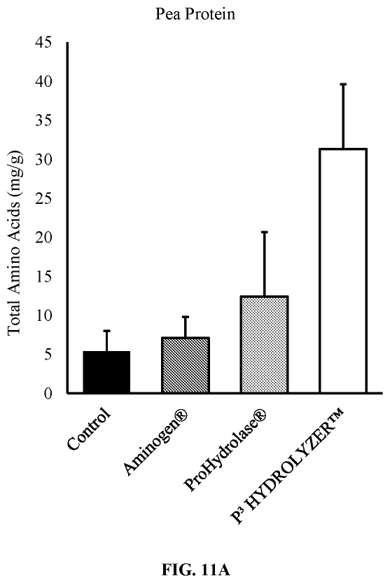

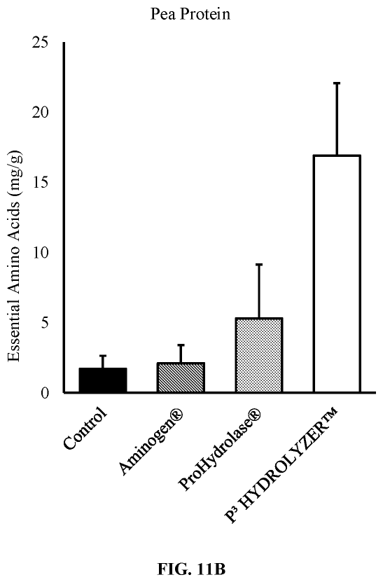

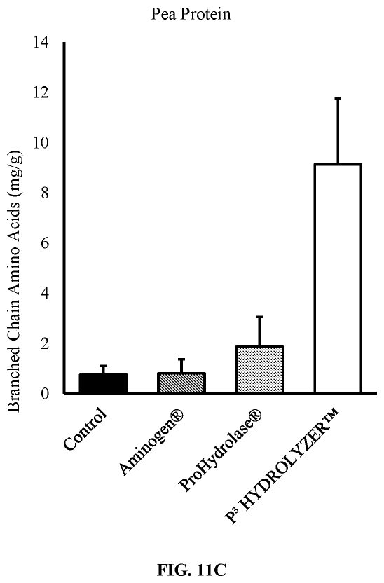

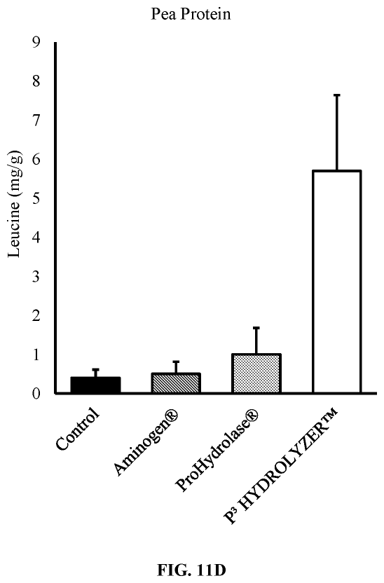

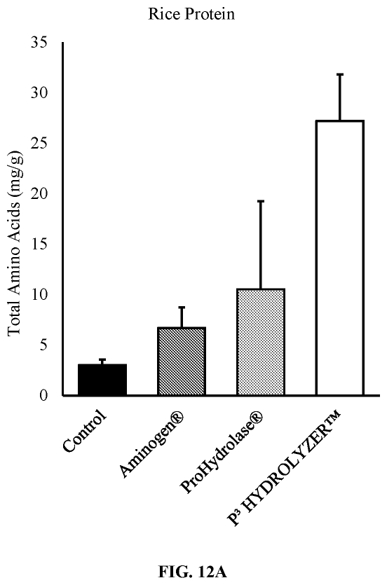

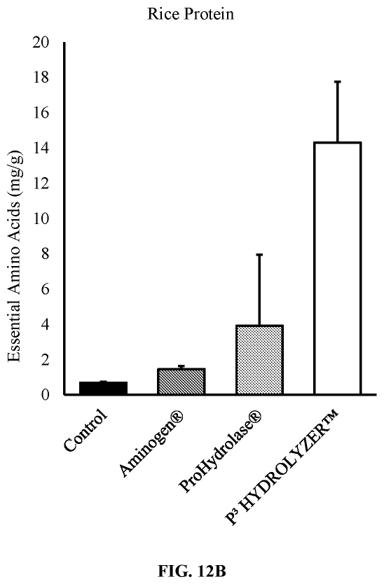

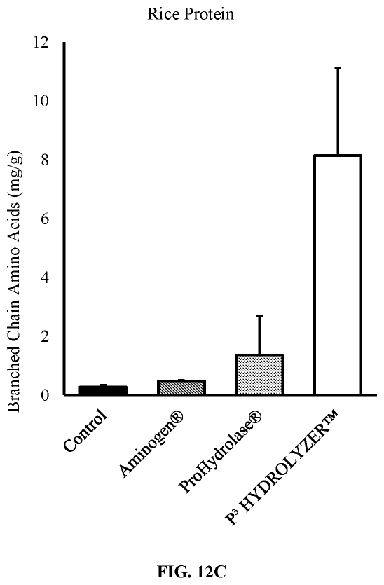

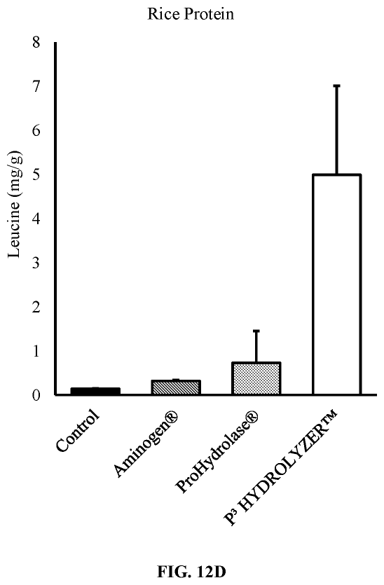

P³ HYDROLYZER™ treatment of soy protein (FIG. 10, Table 8), pea protein (FIG. 11, Table 9), and rice protein (FIG. 12, Table 10) also promoted greater amino acid liberation compared to competitor protease products, including greater liberation of total amino acids, EAA, BCAA, and leucine (all comparisons to P³ HYDROLYZER™ were statistically significant, p<0.05). Altogether, P³ HYDROLYZER™ treatment showed an average of 3-fold greater amino acid liberation and 9-fold greater liberation of BCAA across all three plant protein sources, compared to two top competitors (Table 11).

In this example, whey, soy, pea and rice protein were assayed as protein sources for digestion. However, it is understood that other raw protein sources obtained from plants, fungi, bacteria or animals may also be digested using the protease enzyme mixtures disclosed herein. Similarly, the incubation time and temperature parameters described above may vary as necessary for a given application, while remaining in accordance with the present disclosure.

TABLE 8
Amino acid release from soy protein in a 60 minute simulation of gastric
digestion (Comp, competitor; Comp A, Aminogen ®; Comp B, ProHydrolase ®).

| | | | | | | | |
|---|---|---|---|---|---|---|---|
| Measured | Adjusted | | P³ vs | | | | |
| Value | Values | P³ vs | Comp | Fold | P³ vs Comp | Unadjusted | Unadjusted |
| (mg/g) | (mg/g) | Comp | Average | Change | Unadjusted | average | fold change |

Essential amino acids:

| | | | | | | | | |
|---|---|---|---|---|---|---|---|---|
| Control | 1.5 | | | | | 1667% | | 16x |
| Comp A | 3.1 | 1.6 | 1469% | | | 806% | | |
| Comp B | 5.2 | 3.7 | 635% | 1052% | 10x | 481% | 644% | 6x |
| P³ | 25 | 23.5 | | | | | | |

Branched chain amino acids:

| | | | | | | | | |
|---|---|---|---|---|---|---|---|---|
| Control | 0.59 | | | | | 1820% | | 18x |
| Comp A | 1.09 | 0.5 | 2030% | | | 985% | | |
| Comp B | 1.44 | 0.85 | 1194% | 1612% | 16x | 746% | 866% | 8x |
| P³ | 10.74 | 10.15 | | | | | | |

Total amino acids:

| | | | | | | | | |
|---|---|---|---|---|---|---|---|---|
| Control | 5.2 | | | | | 856% | | 8x |
| Comp A | 8.1 | 2.9 | 1355% | | | 549% | | |
| Comp B | 11.8 | 6.6 | 595% | 975% | 9x | 377% | 463% | 4x |
| P³ | 44.5 | 39.3 | | | | | | |

Leucine:

| | | | | | | | | |
|---|---|---|---|---|---|---|---|---|
| Control | 0.37 | | | | | 1800% | | 18x |
| Comp A | 0.81 | 0.44 | 1430% | | | 822% | | |
| Comp B | 0.95 | 0.58 | 1084% | 1257% | 15x | 701% | 762% | 7x |
| P³ | 6.66 | 6.29 | | | | | | |

TABLE 9
Amino acid release from soy protein in a 60 minute simulation of gastric
digestion (Comp, competitor; Comp A, Aminogen ®; Comp B, ProHydrolase ®).

| | | | | | | | |
|---|---|---|---|---|---|---|---|
| Measured | Adjusted | | P³ vs | | | | |
| Value | Values | P³ vs | Comp | Fold | P³ vs Comp | Unadjusted | Unadjusted |
| (mg/g) | (mg/g) | Comp | Average | Change | Unadjusted | average | fold change |

Essential amino acids:

| | | | | | | | | |
|---|---|---|---|---|---|---|---|---|
| Control | 1.7 | | | | | 994% | | 9x |
| Comp A | 2.1 | 0.4 | 3800% | | | 805% | | |
| Comp B | 5.3 | 3.6 | 422% | 2111% | 21x | 319% | 562% | 5x |
| P³ | 16.9 | 15.2 | | | | | | |

Branched chain amino acids:

| | | | | | | | | |
|---|---|---|---|---|---|---|---|---|
| Control | 0.74 | | | | | 1232% | | 12x |
| Comp A | 0.80 | 0.06 | 13967% | | | 1140% | | |
| Comp B | 1.86 | 1.12 | 748% | 7357% | 73x | 490% | 815% | 8x |
| P³ | 9.12 | 8.38 | | | | | | |

Total amino acids:

| | | | | | | | | |
|---|---|---|---|---|---|---|---|---|
| Control | 5.4 | | | | | 580% | | 5x |
| Comp A | 7.1 | 1.7 | 1524% | | | 441% | | |
| Comp B | 12.4 | 7 | 370% | 947% | 9x | 252% | 347% | 3x |
| P³ | 31.3 | 25.9 | | | | | | |

Leucine:

| | | | | | | | | |
|---|---|---|---|---|---|---|---|---|
| Control | 0.4 | | | | | 1425% | | 14x |
| Comp A | 0.5 | 0.1 | 5300% | | | 1140% | | |
| Comp B | 1.0 | 0.6 | 883% | 3092% | 30x | 570% | 855% | 8x |
| P³ | 5.7 | 5.3 | | | | | | |

TABLE 10
Amino acid release from rice protein in a 60 minute simulation of gastric digestion
(Comp A, Aminogen ®; Comp B, ProHydrolase ®).

| | | | | | | | |
|---|---|---|---|---|---|---|---|
| Measured | Adjusted | | P³ vs | | | | |
| Value | Values | P³ vs | Comp | Fold | P³ vs Comp | Unadjusted | Unadjusted |
| (mg/g) | (mg/g) | Comp | Average | Change | Unadjusted | average | fold change |

Essential amino acids:

| | | | | | | | | |
|---|---|---|---|---|---|---|---|---|
| Control | 0.7 | | | | | 2043% | | 20x |
| Comp A | 1.45 | 0.75 | 1813% | | | 986% | | |
| Comp B | 3.91 | 3.21 | 424% | 1119% | 11x | 366% | 676% | 6x |
| P³ | 14.3 | 13.6 | | | | | | |

Branched chain amino acids:

| | | | | | | | | |
|---|---|---|---|---|---|---|---|---|
| Control | 0.27 | | | | | 3015% | | 360x |
| Comp A | 0.48 | 0.21 | 3748% | | | 1696% | | |
| Comp B | 1.36 | 1.09 | 722% | 2235% | 22x | 599% | 1147% | 11x |
| P³ | 8.14 | 7.87 | | | | | | |

Total amino acids:

| | | | | | | | | |
|---|---|---|---|---|---|---|---|---|
| Control | 3.0 | | | | | 907% | | 9x |
| Comp A | 6.7 | 3.7 | 654% | | | 406% | | |
| Comp B | 10.5 | 7.5 | 323% | 488% | 4x | 259% | 333% | 3x |
| P³ | 27.2 | 24.2 | | | | | | |

Leucine:

| | | | | | | | | |
|---|---|---|---|---|---|---|---|---|
| Control | 0.14 | | | | | 3564% | | 35x |
| Comp A | 0.32 | 0.18 | 2694% | | | 1559% | | |
| Comp B | 0.73 | 0.59 | 822% | 1758% | 17x | 684% | 1121% | 11x |
| P³ | 4.99 | 4.85 | | | | | | |

TABLE 11
Average percent increase in percent greater amino acid liberation
from three plant protein sources by P³ HYDROLYZER ™
compared to the average of two competitor products (Aminogen ® and
ProHydrolase ®) in a 60 minute simulation of gastric digestion.

| | Essential | Branched chain | Total | |
|---|---|---|---|---|
| | amino acids | amino acids | amino acids | Leucine |
| Soy | 644% | 866% | 463% | 762% |
| Pea | 562% | 815% | 347% | 855% |
| Rice | 676% | 1147% | 333% | 1121% |
| Average | 627% | 943% | 381% | 913% |

### Example 3: Comparative Amino Acid Releasing Performance of P³ HYDROLYZER™ in the INFOGEST 122 Minute Simulation of Salivary-Gastric Digestion of Whey, Pea, and Soy Proteins

The “INFOGEST” simulation of salivary-gastric digestion was used to test the efficacy of P³ HYDROLYZER™, Aminogen®, and ProHydrolase® on protein digestion in vitro. Protein substrates included each of 1) Avonlac© 282 whey protein concentrate (Glanbia Nutritionals, Kilkenny, Ireland), 2) soy protein isolate (Hard Eight Nutrition LLC, Hendersonville, NV, USA), and 3) NUTRALYS® S85F pea protein isolate (Roquette Freres; Lestrem, France).

The INFOGEST protocol has been extensively described elsewhere (See Minekus, M., et al., “A Standardised Static In Vitro Digestion Method Suitable for Food—An International Consensus.” Food and Function. 2014. 5(6), 1113-1124; Brodkorb, A., et al., “INFOGEST Static In Vitro Simulation of Gastrointestinal Food Digestion.” Nature Protocols. 2019. 14(4), 991-1014, each of which is hereby incorporated by reference in its entirety). The INFOGEST protocol models three phases of digestion: salivary, gastric, and intestinal. The salivary and gastric phases were modeled herein.

The salivary phase proceeded for 2 minutes in a simulated salivary fluid with agitation at 37° C. and neutral pH in the presence of porcine salivary amylase. The gastric phase proceeded by addition of simulated gastric fluid containing porcine pepsin and incubation for 2 hours with agitation at 37° C. at a starting pH of 3. In the experimental groups (“treatments”), a partial dose of experimental enzymes (i.e., P³ HYDROLYZER™, Aminogen®, or ProHydrolase®) based on the partial serving size of the food substrate, was added to the gastric digesta 10 minutes after the start of the gastric phase to mimic the time to dissolution of a vegetarian capsule shell in the human stomach. The control groups contained protein substrate and the endogenous porcine amylase and porcine pepsin in the salivary and gastric phases, respectively, to model human endogenous enzyme activities. Small samples were withdrawn at the end of the 2 hour gastric phase, followed by inactivation of enzymatic activity at 90° C. for 10 minutes. Samples were stored at 4° C. until measurement of all 20 amino acids by HPLC (Chemstation, Revision B.04.01 SP1, Agilent Technologies; Santa Clara, CA, USA). The results reported here are the average of amino acid values measured from 3 digestion experiments.

Statistical analysis was performed using R® version 3.6.2 (R Core Team, 2020). Figures were produced using GraphPad Prism version 9.1.2 for Windows (San Diego, California, USA). A one-way ANOVA analysis was performed to determine significance and an F-statistic of p<0.05 was considered statistically significant. Tukey's Honestly Significant Difference adjustment was used for multiple comparisons using a 95% family-wise confidence level. Normality was assessed by Shapiro-Wilk test on residuals. Homoscedasticity was assessed with the Levene's Test of Equality of Variances. No violations of normality or homoscedasticity were observed. Data below the limit of quantitation was imputed at the lowest standard value. Amino acids resolved as “not detected” were imputed with a value of zero for analytical purposes. Each amino acid, total EAAs, total BCAAs, and total amino acids were analyzed. All observations are expressed as mean±standard deviation. Figures with asterisks indicate increased levels of significance as follow: *, P<0.05; **, P<0.01; *** P<0.001; ****, P<0.0001.

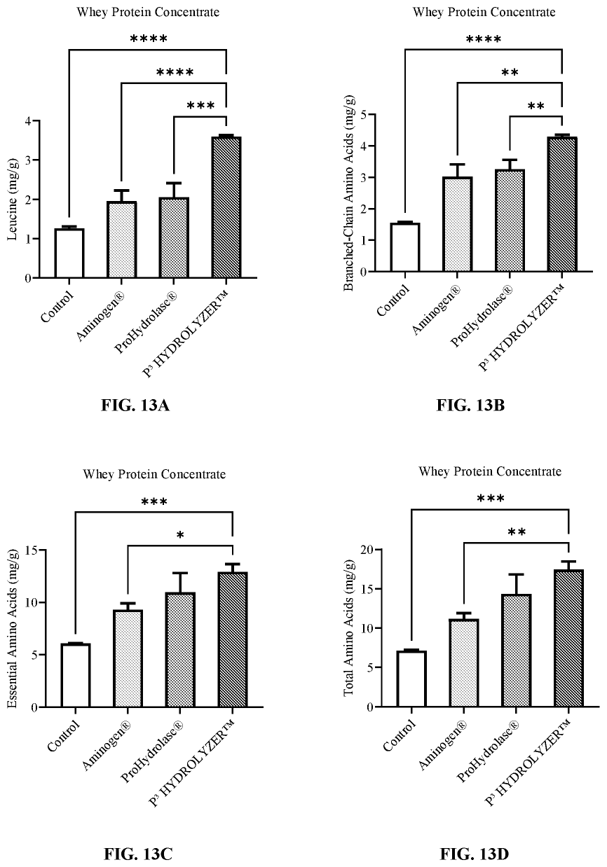

P³ HYDROLYZER™ treatment of whey protein promoted greater amino acid liberation compared to competitor protease products, including greater liberation of leucine, BCAAs, EAAs, and total amino acids leucine (FIG. 13, Table 12). All comparisons to P³ HYDROLYZER™ for leucine and BCAA were significant (p<0.05, FIGS. 13A & 13B). Comparisons to P3 HYDROLYZER™ for EAAs and total amino acids were not statistically significant when compared to ProHydrolase®, but were significant as compared to control and Aminogen® (FIGS. 13C & 13D). The data in Table 12 represent values from averaging the amino acid liberation across 3 separate experiments performed on 3 separate days. Adjusted values are the results of subtracting the porcine enzyme-only control values from the experimental enzyme treatments to establish values normalized to control. Adjusted values are compared vs. P³ HYDROLYZER™ to determine fold-change and percent increases. The “Average” value represents the average of the two competitor enzyme ingredient products, Aminogen® and ProHydrolase©. The “Fold Change” value represents the average of the two competitor enzyme ingredient products, rounded down.

TABLE 12
Amino acid release from whey protein in the INFOGEST
simulation of salivary-gastric digestion.

| | | | | |
|---|---|---|---|---|
| | | | Average | |
| | | % | % | Average |
| Measured | Adjusted | Increase | Increase | Fold |
| Value | Values | (P³ vs | (P³ vs | Change |
| (mg/g) | (mg/g) | Comp) | Comps) | (P³ vs) |

Total amino acids:

| | | | | | |
|---|---|---|---|---|---|
| Control | 7.12 | | | | |
| Aminogen ® | 11.15 | 4.03 | 256% | | |
| ProHydrolase ® | 14.35 | 7.23 | 143% | 200% | 3 |
| P³ | 17.45 | 10.33 | | | |

Essential amino acids:

| | | | | | |
|---|---|---|---|---|---|
| Control | 6.08 | | | | |
| Aminogen ® | 9.29 | 3.21 | 212% | | |
| ProHydrolase ® | 11.00 | 4.92 | 139% | 176% | 2.7 |
| P³ | 12.90 | 6.82 | | | |

Branched chain amino acids:

| | | | | | |
|---|---|---|---|---|---|
| Control | 1.55 | | | | |
| Aminogen ® | 3.02 | 1.47 | 186% | | |
| ProHydrolase ® | 3.26 | 1.71 | 160% | 173% | 2.7 |
| P³ | 4.29 | 2.74 | | | |

Leucine:

| | | | | | |
|---|---|---|---|---|---|
| Control | 1.26 | | | | |
| Aminogen ® | 1.95 | 0.69 | 339% | | |
| ProHydrolase ® | 2.06 | 0.80 | 293% | 316% | 4.1 |
| P³ | 3.60 | 2.34 | | | |

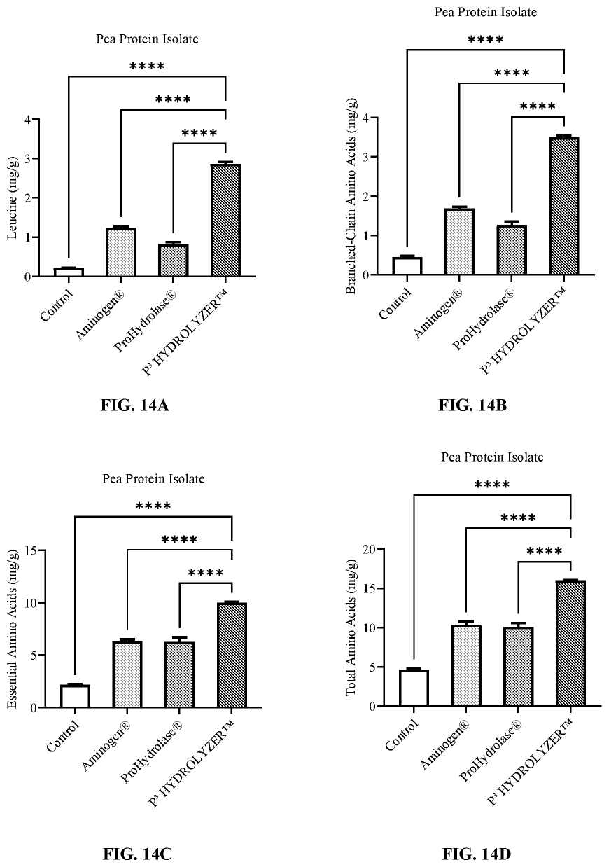

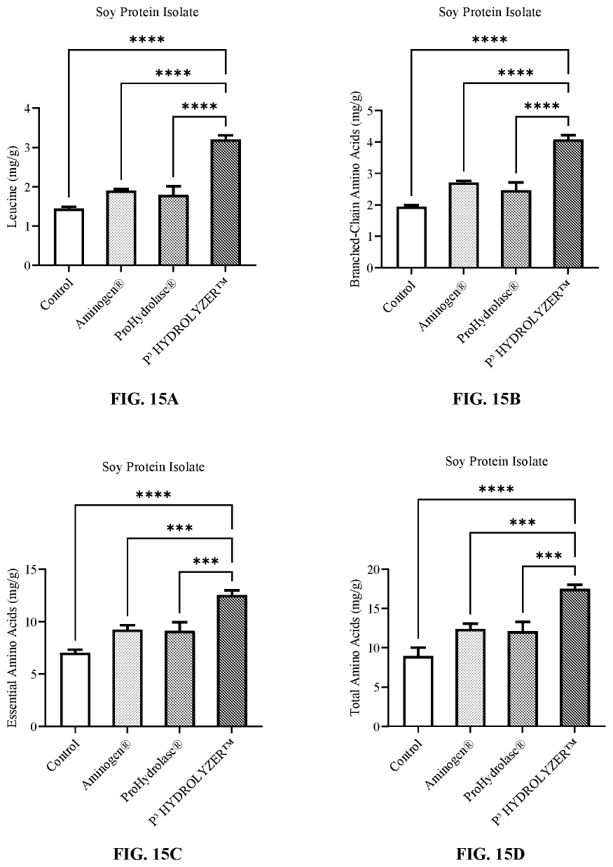

P³ HYDROLYZER™ treatment of pea protein (FIG. 14, Table 13) and soy protein (FIG. 15, Table 14) also promoted greater amino acid liberation compared to competitor protease products, including greater liberation of leucine, BCAAs, EAAs, and total amino acids (all comparisons to P³ HYDROLYZER™ significant, p<0.05). Altogether, P³ HYDROLYZER™ treatment showed an average of two-fold greater total amino acid liberation and 3-fold greater liberation of BCAA across the two plant protein sources, compared to two top competitors (Table 15, FIGS. 14B & 15B).

TABLE 13
Amino acid release from pea protein in the INFOGEST
simulation of salivary-gastric digestion.

| | | | | |
|---|---|---|---|---|
| | | | Average | |
| | | % | % | Average |
| Measured | Adjusted | Increase | Increase | Fold |
| Value | Values | (P³ vs | (P³ vs | Change |
| (mg/g) | (mg/g) | Comp) | Comps) | (P³ vs) |

Total amino acids:

| | | | | | |
|---|---|---|---|---|---|
| Control | 4.61 | | | | |
| Aminogen ® | 10.35 | 5.74 | 198% | | |
| ProHydrolase ® | 10.09 | 5.48 | 208% | 203% | 3 |
| P³ | 15.99 | 11.38 | | | |

Essential amino acids:

| | | | | | |
|---|---|---|---|---|---|
| Control | 2.17 | | | | |
| Aminogen ® | 6.28 | 4.11 | 191% | | |
| ProHydrolase ® | 6.26 | 4.09 | 191% | 191% | 2.9 |
| P³ | 10.00 | 7.83 | | | |

Branched chain amino acids:

| | | | | | |
|---|---|---|---|---|---|
| Control | 0.45 | | | | |
| Aminogen ® | 1.69 | 1.24 | 246% | | |
| ProHydrolase ® | 1.26 | 0.81 | 377% | 311% | 4.1 |
| P³ | 3.50 | 3.05 | | | |

Leucine:

| | | | | | |
|---|---|---|---|---|---|
| Control | 0.22 | | | | |
| Aminogen ® | 1.23 | 1.01 | 261% | | |
| ProHydrolase ® | 0.82 | 0.60 | 440% | 351% | 4.5 |
| P³ | 2.86 | 2.64 | | | |

TABLE 14
Amino acid release from soy protein in the INFOGEST
simulation of salivary-gastric digestion.

| | | | | |
|---|---|---|---|---|
| | | | Average | |
| | | % | % | Average |
| Measured | Adjusted | Increase | Increase | Fold |
| Value | Values | (P³ vs | (P³ vs | Change |
| (mg/g) | (mg/g) | Comp) | Comps) | (P³ vs) |

Total amino acids:

| | | | | | |
|---|---|---|---|---|---|
| Control | 8.94 | | | | |
| Aminogen | 12.40 | 3.46 | 247% | | |
| ProHydrolase | 12.09 | 3.15 | 271% | 259% | 3.5 |
| P³ | 17.48 | 8.54 | | | |

Essential amino acids:

| | | | | | |
|---|---|---|---|---|---|
| Control | 7.03 | | | | |
| Aminogen | 9.24 | 2.21 | 249% | | |
| ProHydrolase | 9.12 | 2.09 | 264% | 256% | 3.5 |
| P³ | 12.54 | 5.51 | | | |

Branched chain amino acids:

| | | | | | |
|---|---|---|---|---|---|
| Control | 1.95 | | | | |
| Aminogen | 2.71 | 0.76 | 280% | | |
| ProHydrolase | 2.47 | 0.52 | 410% | 345% | 4.4 |
| P³ | 4.08 | 2.13 | | | |

Leucine:

| | | | | | |
|---|---|---|---|---|---|
| Control | 1.44 | | | | |
| Aminogen | 1.9 | 0.46 | 385% | | |
| ProHydrolase | 1.79 | 0.35 | 506% | 445% | 5.4 |
| P³ | 3.21 | 1.77 | | | |

TABLE 15
Average percent increase in percent greater amino
acid liberation from 2 plant protein sources by
P³ HYDROLYZER ™ compared to the average
of two competitor products (Aminogen ® and
ProHydrolase ®) in the INFOGEST simulation
of salivary-gastric digestion.

| | Total | Essential | Branched chain | |
|---|---|---|---|---|
| | amino acids | amino acids | amino acids | Leucine |
| Pea | 103% | 91% | 211% | 251% |
| Soy | 159% | 156% | 245% | 345% |
| Average | 131% | 124% | 228% | 298% |

### Example 4: Evaluation of the Organoleptic Properties of an Exemplary Hydrolysate Produced Using P³ HYDROLYZER™

A sensory test was performed in order to determine the effects of the addition of P³ HYDROLYZER™ and ProHydrolase® on sensory attributes of a whey protein shake. The panel of sensory assessors consisted of thirteen volunteers, and the whey protein shakes used in this test were prepared with whey protein powder from NAKED Nutrition® (Coral Gables, FL, USA) and whole milk, as summarized by Table 16.

TABLE 16
Composition of the whey protein shakes for sensory testing.

| | Control | P³ HYDROLYZER ™ | ProHydrolase ® |
|---|---|---|---|
| Whole milk (mL) | 250 | 250 | 250 |
| Whey protein (g) | 25 | 25 | 25 |
| Enzyme (mg) | N/A | 250 | 250 |

Each experimental group was coded with a 3 digit number to keep the study participants blinded. The control samples were numbered 200 to 300, samples treated with P³ HYDROLYZER™ were numbered 400 to 500, and samples treated with ProHydrolase® were numbered 600 to 700. Each assessor evaluated each shake using a rating system based on a 1 to 5 point scale (1, very poor; 2, poor; 3, fair; 4, good; 5, excellent) where a 1 indicated the very poor taste, texture, or overall flavor perception and a 5 indicated excellent taste, texture, or overall flavor perception. Participants also scored one of the 3 experimental shakes as “Best” and one of the remaining two experimental shakes as “Worse.” The results were ranked, scored and then analyzed to determine the preference of protein shakes without enzyme, with P³ HYDROLYZER™, or with ProHydrolase®.

TABLE 17
Sensory testing of whey protein shakes
without added enzymes (control).

| Control | | | | | | |
|---|---|---|---|---|---|---|
| Product | Participant | | | | | |
| Code | ID | Taste | Texture | Overall | Best | Worse |
| 301 | J1 | 4 | 4 | 4 | X | |
| 304 | M3 | 3 | 4 | 4 | X | |
| 310 | J2 | 4 | 5 | 4 | X | |
| 319 | S2 | 3 | 4 | 4 | | |
| 322 | D2 | 4 | 5 | 5 | X | |
| 330 | S1 | 4 | 5 | 4 | X | |
| 350 | K1 | 3 | 5 | 4 | X | |
| 381 | M1 | 3 | 3 | 3 | X | |
| 236 | J3 | 4 | 4 | 4 | | |
| 240 | D1 | 3 | 5 | 5 | | |
| 291 | M2 | 2 | 4 | 3 | | |
| 293 | C1 | 4 | 5 | 4 | X | |
| 295 | K2 | 4 | 4 | 4 | X | |

| | | | | | |
|---|---|---|---|---|---|
| Totals: | 45 | 57 | 52 | 9 | 0 |
| % of Groups: | | | | 69.2% | 0.0% |

TABLE 18
Sensory testing of whey protein shakes
with added P³ HYDROLYZER ™.

| P³ | Partic- | | | | | |
|---|---|---|---|---|---|---|
| HYDROLYZER ™ | ipant | | Tex- | | | |
| Product Code | ID | Taste | ture | Overall | Best | Worse |
| 405 | M2 | 4 | 4 | 4 | | |
| 414 | S1 | 4 | 4 | 4 | | |
| 415 | K1 | 2 | 3 | 2 | | X |
| 434 | M3 | 2 | 3 | 2 | | |
| 447 | S2 | 4 | 5 | 5 | X | |
| 451 | J2 | 4 | 5 | 4 | | |
| 455 | D2 | 5 | 4 | 4 | | |
| 457 | J1 | 2 | 3 | 2 | | X |
| 500 | J3 | 4 | 4 | 4 | | |
| 507 | K2 | 4 | 3 | 3 | | |
| 512 | D1 | 4 | 3.5 | 4 | X | |
| 527 | C1 | 3 | 5 | 4 | | |
| 549 | M3 | 4 | 4 | 4 | X | |

| | | | | | |
|---|---|---|---|---|---|
| Totals: | 46 | 50.5 | 46 | 3 | 2 |
| % of Groups: | | | | 23.1% | 15.4% |

TABLE 19
Sensory testing of whey protein shakes
with added ProHydrolase ®.

| ProHydrolase ® | Participant | | | | | |
|---|---|---|---|---|---|---|
| Product Code | ID | Taste | Texture | Overall | Best | Worse |
| 602 | K1 | 2 | 2 | 2 | | |
| 608 | J2 | 3 | 3 | 3 | | X |
| 622 | M2 | 2 | 2 | 2 | | X |
| 628 | S2 | 2 | 2 | 2 | | X |
| 632 | S1 | 3 | 2 | 3 | | X |
| 638 | D2 | 2 | 1 | 2 | | X |
| 658 | M3 | 1 | 1 | 1 | | X |
| 681 | J1 | 3 | 1 | 2 | | |
| 691 | J3 | 2 | 2 | 2 | | |
| 716 | K2 | 2 | 1 | 2 | | X |
| 747 | DI | 3 | 3 | 3 | | X |
| 750 | M2 | 1 | 1 | 1 | | X |
| 756 | C1 | 2 | 1 | 2 | | X |

| | | | | | |
|---|---|---|---|---|---|
| Totals: | 28 | 22 | 27 | 0 | 10 |
| % of Groups: | | | | 0.0% | 76.9% |

As illustrated by these results shown in Tables 17-19 and FIG. 16, the participants in this blinded experiment generally preferred the protein shake without enzymes, but also preferred the protein shake treated with P³ HYDROLYZER™ rather than an otherwise identical protein shake treated with ProHydrolase®. While this experiment evaluated flavor preferences in connection with a beverage produced using whey protein, it is understood that P³ HYDROLYZER™ may be used to produce protein shakes with desirable flavor profiles from various other protein sources (e.g., plant proteins such as soy, rice, pea, chickpea, or wheat protein, mushroom protein, animal protein, dairy protein, egg protein, bacterial protein, and combinations thereof).

### Example 5: Clinical Evaluation of the Effects of Protease Supplementation on Post-Prandial Plasma Amino Acid Levels

Dietary protein is digested in the stomach and intestines to smaller peptides and 20 individual amino acids which, when absorbed by the gut into circulation and taken up by skeletal muscle, help stimulate muscle protein synthesis (MPS). Amino acids also provide the building blocks for muscle proteins that contribute to muscle growth and increased strength following resistance exercise. Therefore, strategies to efficiently maximize amino acid exposure without protein overconsumption are warranted. Oral enzyme supplementation is a candidate approach to optimize amino acid absorption from dietary protein and protein supplements. Microbial proteases can theoretically speed up the conversion of protein and peptides to amino acids. Protease supplements have been marketed to promote muscle strength by optimizing amino acid absorption, however meaningful and statistically significant clinical evidence is lacking.

Protease supplementation using the proteolytic enzyme mixture described herein was evaluated in a randomized, double-blind, placebo-controlled, cross-over clinical trial. Eligible participants were randomized into groups given either P³ HYDROLYZER™ (n=12) or placebo (n=12) in conjunction with a liquid protein blend for their 1^{st} aminoacidemia trial. A 2^{nd} aminoacidemia trial consisted of the opposite treatment condition. The objective of this study was to determine whether P³ HYDROLYZER™ enzyme supplementation (e.g., with 31,875 HUT protease activity) is an effective dosage to improve the early (0-2 h) and cumulative (0-5 h) net area under the total amino acid, EAA, BCAA, and leucine curves after the ingestion of a protein shake in healthy subjects. Statistically significant results showed that P³ HYDROLYZER™ increased postprandial amino acid concentrations greater than that of the placebo in the acute protein shake challenge aminoacidemia trial. These clinical trial data provide evidence that P³ HYDROLYZER™ supports digestive health, improves protein digestion, and enhances amino acid absorption from dietary protein.

Twenty-four recreationally active healthy adults volunteered to participate in this study. All participants were recruited and performed two experimental trials in the Nutrition and Exercise Performance Research Group clinical laboratory at the University of Illinois at Urbana-Champaign from April to August 2021. All participants were deemed healthy based on their responses to a routine medical screening questionnaire. All participants were informed about the experimental procedures, the purpose of the study, and potential risks before giving written consent. All trials conformed to standards for the use of human participants in research as outlined in the Helsinki Declaration (Clinicaltrials.gov ID NCT04821557) and were approved by the local Institutional Review Board at the University of Illinois at Urbana-Champaign (IRB approval no. 21545)

At Visit 1, study participants were asked to report to the testing facility at ˜0700 hours after an overnight fast for measurement of body weight, height, and body composition by dual-energy x-ray absorptiometry (DEXA; Horizon W, Hologic Inc., Marlborough, MA, USA). After the preliminary testing session, participants were randomized to ingest 25 grams of pea protein isolate (Roquette NUTRALYS® S85F Pea Protein; 101 kcal, 20 g protein, 2.2 g fat) with either added P³ HYDROLYZER or placebo in a counterbalanced fashion on their first experimental trial. For allocation of the participants, a computer-generated list of random numbers was used. The study product was coded with a random numerical-alphabetical code and unblinded after all analyses were completed. Participants were instructed to refrain from any strenuous physical exercise 72 hours and alcohol 48 hours prior to the experimental trial. Participants were provided an identical standardized meal for consumption the evening before both trials (25-30% of energy requirement; 50% of energy from carbohydrate 25% energy from protein and 25% energy from fat). In addition, participants were instructed to maintain the same dietary intake for three days prior to each experimental trial, which was confirmed by the Automated Self-Administered 24-hour (ASA24) Dietary Assessment Tool (version 2020; National Cancer Institute, Bethesda, MD, USA). Both test days were separated by a 7-d wash-out.

For both trials, participants reported to the laboratory between 0600 hours and 0800 hours after an overnight fast. Following assessment of blood pressure, a Teflon catheter was inserted into an antecubital vein, and an arterialized baseline blood sample was collected. Immediately after catheter placement and blood sample collection, participants consumed 25 g of pea protein powder dissolved in 300 mL of water with either P³ HYDROLYZER™ or a maltodextrin placebo. The test articles were manufactured in capsule form, i.e., as: 250 mg P³ HYDROLYZER™ formulated to contain no less than 31,875 HUT activity, plus 20 mg microcrystalline cellulose, or placebo with 250 mg maltodextrin and 20 mg microcrystalline cellulose. On the morning of a protein shake challenge test and aminoacidemia trial, a capsule was opened and contents mixed into the shake. Arterialized blood samples were collected in EDTA-containing tubes before (t=−5 min) and after treatment ingestion (t=15, 30, 45, 60, 75, 90, 120, 150, 180, 210, 240 and 300 min). Blood samples were centrifuged at 3000 g for 10 min at 4° C. and the plasma was subsequently aliquoted and stored at −80° C. for future analysis. Following an at least 1 week washout, participants repeated the aminoacidemia trial at Visit 2 with the opposite test article.

Plasma amino acid concentrations were determined as follows: the Amino Acid standard solution (AAS18, Sigma, USA), containing 2.5 μmol/mL each of L-alanine, L-arginine, L-aspartic acid, L-glutamic acid, glycine, L-histidine, L-isoleucine, L-leucine, L-lysine HCl, L-methionine, L-phenylalanine, L-proline, L-serine, L-threonine, L-tyrosine and L-valine, and 1.25 μmoL/mL L-cystine and a custom mixture containing 2.5 μmol/mL each of L-tryptophane, L-glutamine, L-asparagine, L-citrulline, L-cysteine were used for the calibration curve. Plasma samples (50 μL) were deproteinized with methanol (940 μL), centrifuged with following supernatant evaporation in vacuum and re-suspended in 1 mL of 0.1% formic acid in water before instrument injection. Ten μL of internal standard (DL-p-Chlorophenylalanine, 1 mg/mL 0.1 M HCL) was added to each sample and standard solution. Samples were analyzed by the Thermo Altis Triple Quadrupole liquid chromatography with tandem mass spectrometry (LC/MS/MS) system. Software TraceFinder 4.1 was used for data acquisition and analysis. The LC separation was performed on a Thermo Accucore Vanquish C18+ column (2.1×100 mm, 1.5 μm) with mobile phase A (0.1% formic acid in water) and mobile phase B (0.1% formic acid in acetonitrile) and the flow rate was 0.2 mL/min. The linear gradient was as follows: 0-0.5 min, 0% B; 0.5-3.5 min, 60% B; 3.5-5.5 min, 100% B; 5.5-7.5 min, 0% B. The autosampler and HPLC column chamber were set at 10° C., 50° C., respectively. The injection volume was 1 μL. Mass spectra was acquired under positive electrospray ionization (ESI) with the ion spray voltage of 3500 V. Selected reaction monitoring (SRM) used for the amino acid quantitation.

All data are presented as mean±standard deviation (SD). A priori power analysis was conducted using GPOWER version 3.1.9.2. Based on previous research, the power analysis showed that a sample size of 20 participants was sufficient to detect differences in postprandial AUC BCAA concentrations between conditions when using a one-tailed t-test (p<0.05, 85% power, d=0.62). Accounting for a dropout rate of 15%, we recruited a total of 24 participants. All data were assessed for normality before analysis via visual inspection of normal Q-Q plots and skewness kurtosis values. Differences in amino acid concentrations were analyzed using linear mixed-effects models with time and group as fixed factors and participant intercept as a random effect. Bonferroni's post hoc test was used when significant main effects were identified. Differences in plasma AUC amino acid concentrations were analyzed using a paired samples two-tailed t-test. The level of statistical significance was set at p<0.05 for all analyses. All analyses were performed using IBM SPSS Statistics 23.0 (IBM Corporation, Armonk, NY, USA). For the analysis of amino acid concentrations at baseline, 45, and 60 minute time points, normality was assessed by the Shapiro-Wilk test on residuals and by visual confirmation using QQ-plot. Data points were considered outliers and subsequently removed only if their presence prevented normality. Normality was reassessed prior to removing any additional outliers.

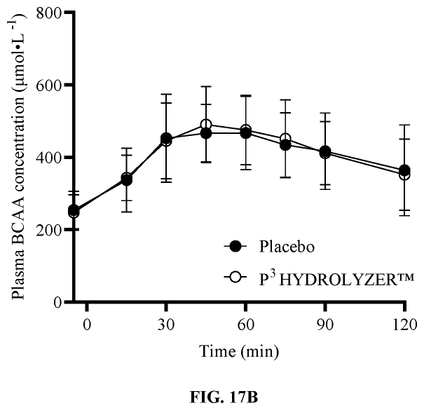

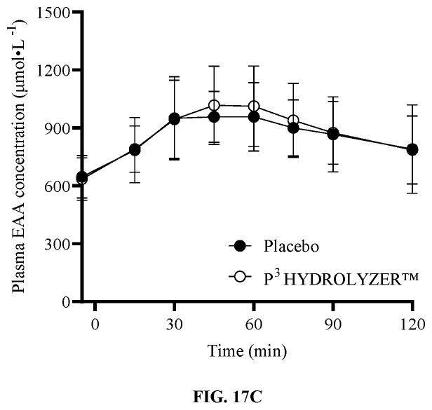

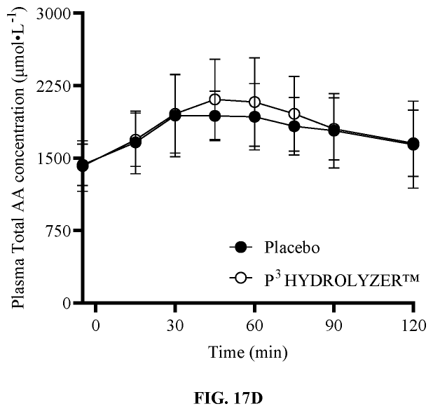

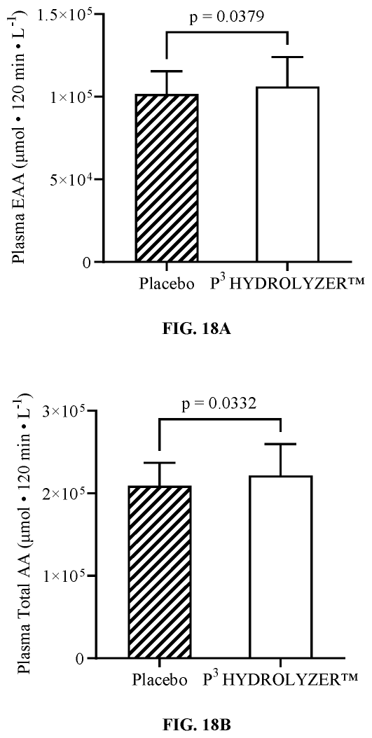

Table 19 reports the subject characteristics at screening. Postprandial leucine, BCAA, EAA, and total amino acid concentrations over time are illustrated in FIG. 17. Postprandial EAA and total amino acid concentrations AUC two hours following consumption of the protein shake are illustrated in FIG. 18. Postprandial leucine, BCAA, EAA and total amino acid concentrations AUC two hours following consumption of the protein shake, with outputs of statistical analysis, are shown in Table 20. Postprandial leucine, BCAA, EAA and total amino acid concentrations at 45 and 60 minutes, absolute and baseline-adjusted, with outputs of statistical analysis are shown in Table 21.

Plasma leucine concentrations increased during the two hours after P³ HYDROLYZER™ and placebo ingestion with the protein shake (time effect: p<0.001), with no significant differences between conditions (condition effect: p=0.602, linear mixed-effects model, FIG. 17A). Plasma leucine concentration AUC showed a trend for near-significantly higher levels during the first two hours with P³ ingestion, compared to placebo (p=0.086, FIG. 18, Table 20). At 60 minutes postprandial, baseline-adjusted plasma leucine concentrations were 13.5% higher with P³ ingestion, compared to placebo (p=0.036, Table 21).

TABLE 19
Subject characteristics at screening.
Subject characteristics at screening

| | | Overall |
|---|---|---|
| Characteristic | Statistic/Category | (n = 24) |
| Biological sex | Female (n) | 12 |
| | Male (n) | 12 |
| Age (years) | Mean | 27 |
| | SD | 4 |
| Weight (kg) | Mean | 74.6 |
| | SD | 11.2 |
| BMI (kg/m²) | Mean | 24.8 |
| | SD | 1.9 |
| BMI group | 18.0-24.99 kg/m² | 13 |
| | 25.0-29.99 kg/m² | 11 |
| Systolic BP (mmHg) | Mean | 119 |
| | SD | 12 |
| Diastolic BP (mmHg) | Mean | 67 |
| | SD | 8 |
| Body fat (%) | Mean | 29.2 |
| | SD | 9.4 |
| Lean body mass | Mean | 50.4 |
| | SD | 13.5 |
| Fasting blood glucose (mmol/L) | Mean | 4.90 |
| | SD | 0.52 |
| Energy intake (kcal/d) | Mean | 2189 |
| | SD | 595 |
| Relative protein intake | Mean | 1.5 |
| (g × kg bw⁻¹ × d⁻¹) | SD | 0.6 |
| Carbohydrate intake (g/d) | Mean | 236 |
| | SD | 69 |
| Fat intake (g/d) | Mean | 93 |
| | SD | 27 |

SD, standard deviation; bw, body weight

Postprandial plasma BCAA concentrations increased during the two hours after P³ HYDROLYZER™ and placebo ingestion with the protein shake (time effect: p<0.001), with no differences between conditions (condition effect: p=0.724, linear mixed-effects model, FIG. 17B). Plasma BCAA concentrations AUC did not differ significantly between conditions during first two hours (p=0.350, Table 20). At 60 minutes postprandial, baseline-adjusted plasma BCAA concentrations were 10.1% higher with P³ ingestion, compared to placebo, albeit not significant (p=0.135, Table 21).

TABLE 20
Postprandial plasma amino acid concentration total area under the curve (AUC) across the first
two hours following consumption of a pea protein shake with placebo or P³ HYDROLYZER ™

| | Amino Acid | | | P³ |
|---|---|---|---|---|
| Timepoint | Category | Statistic | Placebo | HYDROLYZER ™ |
| 0-2 | Leucine | n | 20 | 20 |
| hours | | Mean, μmol · 120 min · L⁻¹ (SD) | 18031 (3326) | 18566 (3133) |
| | | Difference (P³ - Placebo) | 535.3 | |
| | | paired t-test p-value | 0.0858 | |
| | | n | 19 | 19 |
| | BCAA | Mean, μmol · 120 min · L⁻¹ (SD) | 46765 (6559) | 47433 (6674) |
| | | Difference (P³ - Placebo) | 667.8 | |
| | | paired t-test p-value | 0.3497 | |
| | | n | 21 | 21 |
| | EAA | Mean, μmol · 120 min · L⁻¹ (SD) | 101420 (14282) | 103993 (15028) |
| | | Difference (P³ - Placebo) | 2574 | |
| | | paired t-test p-value | 0.0379 | |
| | | n | 21 | 21 |
| | TAA | Mean, μmol · 120 min · L⁻¹ (SD) | 208597 (28265) | 216999 (32284) |
| | | Difference (P³ - Placebo) | 8402 | |
| | | paired t-test p-value | 0.0332 | |

SD, standard deviation

Plasma EAA concentrations increased during the two hours after P³ HYDROLYZER™ and placebo ingestion with the protein shake (time effect: p<0.001), with no differences between conditions (condition effect: p=0.125, linear mixed-effects model, FIG. 17C). Postprandial plasma EAA concentration AUC was significantly greater in the P³ HYDROLYZER™ group when compared to placebo during the first two hours (p=0.038, FIG. 18A, Table 20). At 60 minutes postprandial, baseline-adjusted plasma EAA concentrations were significantly 21.7% higher with P³ ingestion, compared to placebo (p=0.015, Table 21).

Plasma total amino acids concentrations increased during the two hours after protein shake consumption (p<0.001), with higher concentrations with P³ HYDROLYZER™ when compared to placebo (condition effect: p=0.003, linear mixed-effects model, FIG. 17D). Plasma total amino acid concentration AUC was significantly greater in P³ HYDROLYZER™ when compared to placebo during the first two hours (p=0.033, FIG. 18B, Table 20). At 45 and 60 minutes postprandial, baseline-adjusted plasma total amino acid concentrations were significantly 23.5% and 33.0% higher, respectively, with P³ ingestion, compared to placebo (p=0.023 and p=0.005, respectively, Table 21).

TABLE 21
Postprandial plasma amino acid concentrations at 45 and 60 minutes following
consumption of a pea protein shake with P³ HYDROLYZER ™ or placebo.

| | Amino Acid | | | P³ |
|---|---|---|---|---|
| Timepoint | Category | Statistic | Placebo | HYDROLYZER ™ |
| 45 minutes | Leucine | n | 24 | 24 |

| | | | |
|---|---|---|---|
| Mean, μmol/L (SD) | 183.1 (31.75) | 188.7 | (38.70) |

| | | | |
|---|---|---|---|
| | Δ, μmol/L (P³ - Placebo) | 5.64 | |
| | paired t-test p-value | 0.3346 | |
| | Baseline-adjusted comparison^{a} | 6.5% | |
| | paired t-test p-value | 0.2343 | |
| BCAA | n | 23 | 23 |

| | | | |
|---|---|---|---|
| Mean, μmol/L (SD) | 471.4 (77.03) | 482.2 | (99.99) |

| | | | |
|---|---|---|---|
| | Δ, μmol/L (P³ - Placebo) | 10.76 | |
| | paired t-test p-value | 0.4601 | |
| | Baseline-adjusted comparison^{a} | 6.5% | |
| | paired t-test p-value | 0.2731 | |
| EAA | n | 23 | 23 |

| | | | |
|---|---|---|---|
| Mean, μmol/L (SD) | 965.7 (128.1) | 999.7 | (188.1) |

| | | | |
|---|---|---|---|
| | Δ, μmol/L (P³ - Placebo) | 34.03 | |
| | paired t-test p-value | 0.1989 | |
| | Baseline-adjusted comparison^{a} | 12.7% | |
| | paired t-test p-value | 0.1040 | |
| TAA | n | 22 | 22 |

| | | | |
|---|---|---|---|
| Mean, μmol/L (SD) | 1952 (258.5) | 2019 | (337.7) |

| | | | | |
|---|---|---|---|---|
| | | Δ, μmol/L (P³ - Placebo) | 157.3 | |
| | | paired t-test p-value | 0.0009 | |
| | | Baseline-adjusted comparison^{a} | 23.5% | |
| | | paired t-test p-value | 0.0225 | |
| 60 minutes | Leucine | n | 23 | 23 |

| | | | |
|---|---|---|---|
| Mean, μmol/L (SD) | 175.2 (36.37) | 185.4 | (39.03) |

| | | | |
|---|---|---|---|
| | Δ, μmol/L (P³ - Placebo) | 10.14 | |
| | paired t-test p-value | 0.1056 | |
| | Baseline-adjusted comparison^{a} | 13.5% | |
| | paired t-test p-value | 0.0361 | |
| BCAA | n | 24 | 24 |

| | | | |
|---|---|---|---|
| Mean, μmol/L (SD) | 466.8 (100.90) | 479.9 | (96.86) |

| | | | |
|---|---|---|---|
| | Δ, μmol/L (P³ - Placebo) | 13.06 | |
| | paired t-test p-value | 0.3525 | |
| | Baseline-adjusted comparison^{a} | 10.1% | |
| | paired t-test p-value | 0.1351 | |
| EAA | n | 24 | 24 |

| | | | |
|---|---|---|---|
| Mean, μmol/L (SD) | 957.1 (177.6) | 1012 | (2074.6) |

| | | | |
|---|---|---|---|
| | Δ, μmol/L (P³ - Placebo) | 55.33 | |
| | paired t-test p-value | 0.0448 | |
| | Baseline-adjusted comparison^{a} | 21.7% | |
| | paired t-test p-value | 0.0153 | |
| TAA | n | 24 | 24 |

| | | | |
|---|---|---|---|
| Mean, μmol/L (SD) | 1927 (342.5) | 2079 | (457.2) |

| | |
|---|---|
| Δ, μmol/L (P³ - Placebo) | 152.1 |
| paired t-test p-value | 0.017 |
| Baseline-adjusted comparison^{a} | 33.0% |
| paired t-test p-value | 0.0053 |

^{a}Average baseline amino acid concentration was subtracted from average amino acid concentration at each time point, within each experimental group, and then compared between groups and expressed as a relative percent increase (P3 vs Placebo). n, sample size; SD, standard deviation; Δ, difference

This is the first randomized, double-blind, placebo-controlled, cross-over study showing the efficacy of microbial protease supplementation in increasing postprandial plasma amino acid concentrations following protein shake consumption in healthy men and women. The results suggest that P³ HYDROLYZER™ is a well-tolerated and safe strategy to improve dietary protein digestion and increase postprandial plasma concentrations in healthy adults.

A study according to this exemplary protocol may be used to evaluate the effects of protease supplementation using the proteolytic enzyme mixtures described herein and to provide data that can be used to select optimal amounts and/or administration schedules for the proteolytic enzyme mixtures described herein.

Description of Sequences

| | |
|---|---|
| SEQ ID No.: | 1 |
| Trade name: | Fungal Protease A (BIO-CAT, Inc.) |
| Source organism: | Aspergillus oryzae |
| Protein: | Aspergillopepsin-1 |
| Gene: | pepA |
| UniPROTKB ID: | Q06902 |
| Synonyms: | Acid protease A2, Aspartic protease A2, Aspergillus acid |
| | protease Aspergillus acid proteinase, Aspergillus aspartic |
| | proteinase, Aspergillopepsin I, Aspergillopepsin O, |
| | Aspergillopeptidase A |
| IUBMB Enzyme | 3.4.23.18 |
| Commission (EC) number: | |
| Length: | ≤404 amino acids |
| Amino acid sequence: | 10 20 30 40 50 |
| | MVILSKVAAV AVGLSTVASA LPTGPSHSPH ARRGFTINQI TRQTARVGPK |
| | 60 70 80 90 100 |
| | TASFPAIYSR ALAKYGGTVP AHLKSAVASG HGTVVTSPEP NDIEYLTPVN |
| | 110 120 130 140 150 |
| | IGGTTLNLDF DTGSADLWVF SEELPKSEQT GHDVYKPSGN ASKIAGASWD |
| | 160 170 180 190 200 |
| | ISYGDGSSAS GDVYQDTVTV GGVTAQGQAV EAASKISDQF VQDKNNDGLL |
| | 210 220 230 240 250 |
| | GLAFSSINTV KPKPQTTFFD TVKDQLDAPL FAVTLKYHAP GSYDFGFIDK |
| | 260 270 280 290 300 |
| | SKFTGELAYA DVDDSQGFWQ FTADGYSVGK GDAQKAPITG IADTGTTLVM |
| | 310 320 330 340 350 |
| | LDDEIVDAYY KQVQGAKNDA SAGGYVFPCE TELPEFTVVI GSYNAVIPGK |
| | 360 370 380 390 400 |
| | HINYAPLQEG SSTCVGGIQS NSGLGLSILG DVFLKSQYVV FDSQGPRLGF |
| | AAQA |
| Molecular mass: | ≤43 kDa (irrespective of post-translational modifications |
| | and including lower molecular weight transcriptional or |
| | alternate promoter variants) |
| SEQ ID No.: | 2 |
| Trade Name: | Fungal Protease AM (BIO-CAT, Inc.) |
| Protein: | Leucine aminopeptidase 1, variant 2 |
| Gene: | LAP1 |
| NCBI Accession: | XP_045945651 |
| IUBMB Enzyme | 3.4.11.- |
| Commission (EC) | |
| number: | |
| Source organism: | Aspergillus melleus |
| Length: | ≤387 amino acids |
| Amino Acid sequence: | 10 20 30 40 50 |
| | MKVGAALALG ATASTGVLAA VIPQAPLTNP HIYHNQEKYL IELAPYQTRW |
| | 60 70 80 90 100 |
| | VTEEEKWALK LDGVNFIDVT TERNAGFYPT LHAPSYVRYP SKMEHTDEVT |
| | 110 120 130 140 150 |
| | TLIKDLSKAN MQHNLEKFTS FHTRYYKSQT GIESATWLYN QVLDVIKSSG |
| | 160 170 180 190 200 |
| | AAKHGATVDQ FAHPWGQFSV IARVPGKTNK TVVLGAHQDS INLFLPSILA |
| | 210 220 230 240 250 |
| | APGADDDGSG TVTILEALRG LLQSDPIIKG EAPNTIEFHW YSAEEGGMLG |
| | 260 270 280 290 300 |
| | SQAIFSQYKQ DKRDIKAMLQ QDMTGYTQGA LEAGRQEAVG IMVDYVDQGL |
| | 310 320 330 340 350 |
| | TQFLKDAVTT YCDIGFINTK CGYACSDHTS ASKYGYPAAM ATESEMENSN |
| | 360 370 380 |
| | KRIHTTDDKI KYLSFDHMLQ HAKLTLGFAY ELAFAPF |
| Molecular mass: | ≤43 kDa (irrespective of post-translational modifications |
| | and including lower molecular weight transcriptional or |
| | alternate promoter variants) |
| SEQ ID No.: | 3 |
| Trade name: | Fungal Protease A2 (BIO-CAT, Inc.) |
| Source organism: | Aspergillus oryzae |
| Protein: | Extracellular metalloproteinase NpI |
| Gene: | NpI |
| UniPROTKB ID: | Q2U1G7 |
| Synonyms: | Elastinolytic metalloproteinase NpI, |
| | Fungalysin NpI, Neutral protease I |
| IUBMB Enzyme | 3.4.24.- |
| Commission | |
| (EC) number: | |
| Length: | ≤634 amino acids |
| Amino acid sequence: | 10 20 30 40 50 |
| | MRGLLLAGAL GLPLAVLAHP THHAHGLQRR TVDLNSFRLH QAAKYINATE |
| | 60 70 80 90 100 |
| | SSSDVSSSFS PFTEQSYVET ATQLVKNILP DATFRVVKDH YIGSNGVAHV |
| | 110 120 130 140 150 |
| | NFRQTAHGLD ANPLTKRDYT IDNADFNVNV GKNGKIFSYG HSFYTGKIPD |
| | 160 170 180 190 200 |
| | DPVAALRGTN EALQLSITLD QVSTEATEDK ESFNFKGVSG TVSDPKAQLV |
| | 210 220 230 240 250 |
| | YLVKEDGSLA LTWKVETDID SNWLLTYIDA NTGKDVHGVV DYVAEADYQV |
| | 260 270 280 290 300 |
| | YAWGINDPTE GPRTVISDPW DSSASAFTWI SDGENNYTTT RGNNGIAQSN |
| | 310 320 330 340 350 |
| | PTGGSQYLKN YRPDSPDLKF QYPYSLNATP PESYIDASIT QLFYTANTYH |
| | 360 370 380 390 400 |
| | DLLYTLGFNE EAGNFQYDNN GKGGAGNDYV ILNAQDGSGT NNANFATPPD |
| | 410 420 430 440 450 |
| | GQPGRMRMYI WTESQPYRDG SFEAGIVIHE YTHGLSNRLT GGPANSRCLN |
| | 460 470 480 490 500 |
| | ALESGGMGEG WGDFMATAIR LKAGDTHSTD YTMGEWAANK KGGIRAYPFS |
| | 510 520 530 540 550 |
| | TSLETNPLTY TSLNELDEVH AIGAVWANVL YELLWNLIDK HGKNDGPKPE |
| | 560 570 580 590 600 |
| | FKDGVPTDGK YLAMKLVIDG MALQPCNPNC VQARDAILDA DKALTDGANK |
| | 610 620 630 |
| | CEIWKAFAKR GLGEGAEYHA SRRVGSDKVP SDAC |
| Molecular mass: | ≤70 kDa (irrespective of post-translational |
| | modifications and including lower molecular |
| | weight transcriptional or alternate promoter variants) |

1. A proteolytic enzyme mixture comprising a plurality of fungal proteases from members of the genus Aspergillus, wherein the plurality of fungal proteases comprises: (a) a 42 kDa protease with exo- and endo-protease activity obtained from A. oryzae, (b) a 34 kDA protease with peptidase activity obtained from A. melleus, and (c) a 35 kDa fungal neutral protease obtained from A. oryzae, and the proteolytic enzyme mixture has exoprotease, endoprotease and peptidase activity.

2. The proteolytic enzyme mixture of claim 1, wherein the ratio of the 42 kDa protease:34 kDA protease:35 kDa fungal neutral protease is:
a) 10:1:40; or
b) 5-15:0.5-1.5:30-50;
as measured in Hemoglobin Unit Tyrosine base (HUT) units.

3. The proteolytic enzyme mixture of claim 1, wherein the mixture displays peak proteolytic activity at approximately pH 6.

4. The proteolytic enzyme mixture of claim 1, wherein the mixture maintains proteolytic activity across a pH range of approximately 3.0 to 9.0.

5. The proteolytic enzyme mixture of claim 1, wherein the mixture is stable over a temperature range of approximately 20 to 80° C.

6. The proteolytic enzyme mixture of claim 1, wherein the mixture has a relative activity level of at least 40% over a temperature range of approximately 20 to 80° C.

7. A proteolytic enzyme mixture comprising a plurality of fungal proteases from the genus Aspergillus,
wherein the mixture comprises at least two of SEQ ID NOs: 1, 2, and 3; or
wherein the mixture includes at least one variant of SEQ ID NOs: 1, 2, or 3, comprising:
a) an enzyme which shares at least 90, 91, 92, 93, 94, 95, 96, 97, 98 or 99% full-length sequence identity with any one of SEQ ID NOs: 1-3;
b) an enzyme which shares at least 90, 91, 92, 93, 94, 95, 96, 97, 98 or 99% full-length sequence identity with the region spanning position 78-404 of SEQ ID NO: 1;
c) an enzyme which shares at least 90, 91, 92, 93, 94, 95, 96, 97, 98 or 99% full-length sequence identity with the region spanning position 246-634 of SEQ ID NO: 3; or
d) any combination of a), b) and/or c);
wherein the variant retains the enzymatic activity of any one of SEQ ID NOs: 1-3.

8. (canceled)

9. The proteolytic enzyme mixture of claim 1, in a dehydrated, powdered, granular or freeze-dried form.

10. A dietary supplement comprising the proteolytic enzyme mixture of claim 1.

11. A dietary supplement comprising the proteolytic enzyme mixture of claim 1 and at least one additional enzyme comprising:
a) a carbohydrase;
b) an alpha-amylase; and/or
c) an alpha-galactosidase.

12. The dietary supplement of claim 10, wherein the dietary supplement is:
a) a protein supplement or a nutritional supplement; and/or
b) formulated as a tablet, capsule, or powder.

13. The dietary supplement of claim 10, wherein the dietary supplement contains at least 0.04% by weight solids of a protease preparation from Aspergillus oryzae.

14. A method for increasing dietary protein digestion and/or the absorption of amino acids from dietary protein, comprising:
administering a proteolytic enzyme mixture comprising a plurality of fungal proteases from members of the genus Aspergillus, wherein the plurality of fungal proteases comprises: (a) a 42 kDa protease with exo- and endo-protease activity obtained from A. oryzae, (b) a 34 kDA protease with peptidase activity obtained from A. melleus, and (c) a 35 kDa fungal neutral protease obtained from A. oryzae, and the proteolytic enzyme mixture has exoprotease, endoprotease and peptidase activity, or the dietary supplement of claim 10, to a subject at least once per day.

15. The method of claim 14, wherein the dietary supplement is administered at least twice per day, at least three times per day, or at least four times per day.

16. The method of claim 14, wherein the dietary supplement is administered in an amount effective to increase postprandial plasma concentrations of total amino acids, essential amino acids, branched chain amino acids, and/or leucine in the subject.

17. The method of claim 14, wherein the dietary supplement is administered to the subject before, after, or concurrently with a food or beverage comprising at least 10 wt. % of protein.

18. A method for improving muscle health, increasing muscle protein synthesis, increasing muscle size, and/or improving muscle strength, comprising:
administering a proteolytic enzyme mixture comprising a plurality of fungal proteases from members of the genus Aspergillus, wherein the plurality of fungal proteases comprises: (a) a 42 kDa protease with exo- and endo-protease activity obtained from A. oryzae, (b) a 34 kDA protease with peptidase activity obtained from A. melleus, and (c) a 35 kDa fungal neutral protease obtained from A. oryzae, and the proteolytic enzyme mixture has exoprotease, endoprotease and peptidase activity, or the dietary supplement of claim 10, to a subject at least once per day.

19. A method of increasing exercise performance, decreasing muscle breakdown during exercise, and/or improving recovery from exercise comprising:
administering a proteolytic enzyme mixture comprising a plurality of fungal proteases from members of the genus Aspergillus, wherein the plurality of fungal proteases comprises: (a) a 42 kDa protease with exo- and endo-protease activity obtained from A. oryzae, (b) a 34 kDA protease with peptidase activity obtained from A. melleus, and (c) a 35 kDa fungal neutral protease obtained from A. oryzae, and the proteolytic enzyme mixture has exoprotease, endoprotease and peptidase activity, a food product comprising said proteolytic enzyme mixture, or the dietary supplement of claim 10, to a subject in an amount sufficient to increase exercise performance and/or decrease muscle breakdown during exercise.

20. The method of claim 19, wherein the food product is a protein shake.

21. The method of claim 20, wherein the protein shake comprises a protein source from dairy, plant, or a mushroom.


SEQUENCE LISTING (3 sequences)

SEQ ID NO: 1 - protein, 404 aa, Aspergillus oryzae
MVILSKVAAVAVGLSTVASALPTGPSHSPHARRGFTINQITRQTARVGPKTASFPAIYSRALAKYGGTVPAHLKSAVASGHGTVVTSPEPNDIEYLTPVNIGGTTLNLDFDTGSADLWVFSEELPKSEQTGHDVYKPSGNASKIAGASWDISYGDGSSASGDVYQDTVTVGGVTAQGQAVEAASKISDQFVQDKNNDGLLGLAFSSINTVKPKPQTTFFDTVKDQLDAPLFAVTLKYHAPGSYDFGFIDKSKFTGELAYADVDDSQGFWQFTADGYSVGKGDAQKAPITGIADTGTTLVMLDDEIVDAYYKQVQGAKNDASAGGYVFPCETELPEFTVVIGSYNAVIPGKHINYAPLQEGSSTCVGGIQSNSGLGLSILGDVFLKSQYVVFDSQGPRLGFAAQA

SEQ ID NO: 2 - protein, 387 aa, Aspergillus melleus
MKVGAALALGATASTGVLAAVIPQAPLTNPHIYHNQEKYLIELAPYQTRWVTEEEKWALKLDGVNFIDVTTERNAGFYPTLHAPSYVRYPSKMEHTDEVTTLIKDLSKANMQHNLEKFTSFHTRYYKSQTGIESATWLYNQVLDVIKSSGAAKHGATVDQFAHPWGQFSVIARVPGKTNKTVVLGAHQDSINLFLPSILAAPGADDDGSGTVTILEALRGLLQSDPIIKGEAPNTIEFHWYSAEEGGMLGSQAIFSQYKQDKRDIKAMLQQDMTGYTQGALEAGRQEAVGIMVDYVDQGLTQFLKDAVTTYCDIGFINTKCGYACSDHTSASKYGYPAAMATESEMENSNKRIHTTDDKIKYLSFDHMLQHAKLTLGFAYELAFAPF

SEQ ID NO: 3 - protein, 634 aa, Aspergillus oryzae
MRGLLLAGALGLPLAVLAHPTHHAHGLQRRTVDLNSFRLHQAAKYINATESSSDVSSSFSPFTEQSYVETATQLVKNILPDATFRVVKDHYIGSNGVAHVNFRQTAHGLDIDNADFNVNVGKNGKIFSYGHSFYTGKIPDANPLTKRDYTDPVAALRGTNEALQLSITLDQVSTEATEDKESFNFKGVSGTVSDPKAQLVYLVKEDGSLALTWKVETDIDSNWLLTYIDANTGKDVHGVVDYVAEADYQVYAWGINDPTEGPRTVISDPWDSSASAFTWISDGENNYTTTRGNNGIAQSNPTGGSQYLKNYRPDSPDLKFQYPYSLNATPPESYIDASITQLFYTANTYHDLLYTLGFNEEAGNFQYDNNGKGGAGNDYVILNAQDGSGTNNANFATPPDGQPGRMRMYIWTESQPYRDGSFEAGIVIHEYTHGLSNRLTGGPANSRCLNALESGGMGEGWGDFMATAIRLKAGDTHSTDYTMGEWAANKKGGIRAYPFSTSLETNPLTYTSLNELDEVHAIGAVWANVLYELLWNLIDKHGKNDGPKPEFKDGVPTDGKYLAMKLVIDGMALQPCNPNCVQARDAILDADKALTDGANKCEIWKAFAKRGLGEGAEYHASRRVGSDKVPSDAC